(19) United States
(12) Patent Application Publication
(10) Pub. No.: US 2021/0079484 A1
(43) Pub. Date: Mar. 18, 2021

(54) BIOMARKER FOR PREDICTING EQUINE GAIT AND METHODS OF USE THEREOF

(71) Applicant: Regents of the University of Minnesota, Minneapolis, MN (US)

(72) Inventors: Molly Elizabeth MCCUE, Minneapolis, MN (US); Annette Marie MCCOY, Minneapolis, MN (US)

(73) Assignee: Regents of the University of Minnesota, Minneapolis, MN (US)

(21) Appl. No.: 17/048,434

(22) PCT Filed: Apr. 16, 2019
(86) PCT No.: PCT/US2019/027725

§ 371 Date: Oct. 16, 2020

Related U.S. Application Data
(60) Provisional application No. 62/658,809, filed on Apr. 17, 2018.

(51) Int. Cl. C12Q 1/6888; G16B 40/20; G16B 20/20

(52) U.S. Cl. CPC C12Q 1/6888; G16B 40/20; C12Q 2600/156; C12Q 2600/124; G16B 20/20

(57) ABSTRACT

Certain embodiments of the invention provide a method for identifying a horse having the ability to pace, comprising assaying a nucleic acid sample from the horse to detect the genotype of at least one single nucleotide polymorphism (SNP) described herein.

### RELATED APPLICATION

This application claims the benefit of priority of U.S. Provisional Application Ser. No. 62/658,809 filed on Apr. 17, 2018, which application is incorporated by reference herein.

### GOVERNMENT FUNDING

This invention was made with government support under 2012-67015-19432 awarded by the United States Department of Agriculture. The government has certain rights in the invention.

### BACKGROUND OF THE INVENTION

Gait refers to a pattern of limb movement during locomotion, and can be defined by patterns of footfall and symmetry, among other factors. In quadrupeds, a limited number of gaits are conserved among species, including the walk (4-beat, symmetric), trot (two-beat, symmetrical, diagonal), and gallop (4-beat, asymmetric). Deviations from normal gait patterns are suggestive of underlying musculoskeletal or neurologic abnormalities. However, certain breeds of horses, including the Standardbred, Icelandic horse, Tennessee Walking Horse, and Paso Fino, have been specifically selected over generations of breeding for their ability to perform alternative patterns of locomotion. These alternative gaits are typically of intermediate speed and replace the trot. There are strong signatures of selection evident when comparing gaited and non-gaited breeds (Petersen et al., PLoS Genet. 2013; 9(1):e1003211), and the trait is highly heritable; for example, heritabilities of the pace and tölt in the Icelandic horse have been estimated to range between 0.53 and 0.73 (Albertsdottir et al., J Anim Breed Genet. 2011; 128(2):124-32). However, until recently, the specific genetic determinants underlying these alternative gaits were completely unknown.

In 2012, a genome-wide association study (GWAS) in four-gaited (walk, trot, tölt, and gallop) and five-gaited (walk, trot, tölt, gallop, and pace) Icelandic horses revealed a strongly associated SNP on equine (ECA) chromosome 23 (Andersson et al., Nature. 2012; 488(7413):642-6). Deep (30× coverage) whole-genome sequencing of one four-gaited and one five-gaited individual revealed a premature stop codon in the last exon of DMRT3 (an isoform of the doublesex and mab-3 related transcription factor). Subsequent genotyping of additional Icelandic horses revealed that nearly all five-gaited individuals were homozygous for the mutation, compared to only a third of the four-gaited horses. Of even greater interest, when horses of other breeds were genotyped for the mutation, it was found to be nearly fixed in gaited breeds (e.g. Paso Fino, Peruvian Paso, Tennessee Walking Horse, Standardbred), but absent in non-gaited breeds (e.g. Arabian, Thoroughbred) (Andersson et al., Nature. 2012; 488(7413):642-6). The functional importance of DMRT3 was confirmed in a mouse model, where mice null for DMRT3 exhibited an abnormal gait characterized by an increased stride, prolonged, stance and swing phases of both the thoracic and pelvic limbs, and near absence of coordinated pelvic limb movements. Further, DMRT3 expression was localized to the spinal cord both pre- and postnatally, and null mice had fewer commissural interneurons, suggesting that this gene is important for the development of normal locomotor coordination (Andersson et al., Nature. 2012; 488(7413):642-6).

Although the DMRT3 mutation appears to be necessary for “gaitedness” in horses it is not sufficient to explain the variation of this trait, as demonstrated by the fact that it is nearly fixed in Standardbreds, although not all individuals exhibit that breed's alternative gait, pacing (Andersson et al., Nature. 2012; 488(7413):642-6). It is noteworthy that approximately 20% of the offspring of Standardbred trotter stallions go on to race as pacers (Cothran et al., Anim Genet. 1987; 18(4):285-96). It is unknown whether this is due to genetic predisposition, training, or a combination of the two, but it is likely that modifying genetic factors segregate in the Standardbred population and determine an individual's ability to pace.

Thus, there is a need to identify new biomarkers for these alternative gaits.

### SUMMARY OF THE INVENTION

Certain embodiments of the invention provide a method for identifying a horse having the ability to pace, comprising:

1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1; and

2) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

Certain embodiments of the invention provide a method, comprising:

1) obtaining or having obtained a physiological sample from a horse, wherein the physiological sample comprises nucleic acid;

2) assaying or having assayed the sample to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1; and

3) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

Certain embodiments of the invention provide a method for selecting and training a horse for racing, comprising:

1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1;

2) identifying or having identified the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected; and

3) training the identified horse for racing.

Certain embodiments of the invention provide a method for selecting a horse for a breeding program, comprising:

1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1;

2) identifying or having identified the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected; and

3) breeding the identified horse or obtaining a sperm or egg sample from the identified horse.

Certain embodiments of the invention provide a method of detecting at least one genetic variation in a horse, comprising:

1) obtaining or having obtained a physiological sample from the horse, wherein the physiological sample comprises nucleic acid; and

2) assaying the sample to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1.

Certain embodiments of the invention provide a method of measuring a panel of single nucleotide polymorphisms (SNPs) in a horse to predict whether the horse has the ability to pace, comprising:

1) obtaining or having obtained a physiological sample from the horse, wherein the physiological sample comprises nucleic acid;

2) assaying the sample to detect the genotype of each SNP in the panel, wherein the panel comprises two or more SNPs selected from the group consisting of a SNP located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and at position 35726345 on ECA1.

Certain embodiments of the invention provide a nucleic acid comprising a single nucleotide polymorphism described herein.

### BRIEF DESCRIPTION OF THE FIGURES

FIG. 1. Manhattan plot of results from mixed model analysis using GEMMA. The 31 autosomal and X chromosome (32) are represented in different shades along the x-axis and the—log(p-value) is on the y-axis. Each dot represents a SNP. Genome-wide significant hits are on ECA1, 2, 6, 9, 17, 23, 25, and 31. See, Table 1 for specific SNPs and p-values. The top horizontal line represents the level of genome-wide significance (p<1.44×10⁻⁶⁻); the bottom line represents a cutoff for moderate association (p<1×10⁻⁵).

FIG. 2. Conditional inference tree based on genotyping results in 659 Standardbreds with race records. Grey nodes are pacers; black nodes are trotters. Nodes 5, 7, 10, and 15 contain misclassified individuals (total n=6).

FIG. 3. Multi-dimensional scaling (MDS) plot of 542 Standardbred horses (366 trotters, 176 pacers) based on genome-wide genotyping data. The two groups are genetically distinct, with minimal admixture.

### DETAILED DESCRIPTION

Certain breeds have been developed over generations specifically for the ability to perform alternative patterns of movement, or gaits. Current understanding of the genetic basis for these gaits is limited to one known mutation apparently necessary, but not sufficient, for explaining variability in “gaitedness.” The Standardbred breed includes two distinct groups, pacers, which exhibit an alternative two-beat gait (the pace) where the legs on the same side of the body move together, and trotters, which do not exhibit this gait. The long-term objective of the experiments described herein was to identify variants underlying the ability of certain Standardbreds to pace. In this study, several regions of the genome highly associated with gait were identified and, within these regions, a number of specific variants highly associated with gait were identified. Although the biological function of these variants has yet to be determined, a model was developed based on seven variants that was >99% accurate in predicting whether an individual was a pacer or a trotter in two independent populations. This predictive model can be used by horse owners to, e.g., make breeding and training decisions related to this economically important trait, and used by scientists interested in understanding the biology of gait development.

Thus, the present invention provides a method for detecting the presence of at least one biomarker associated with alternative equine gaits (e.g., ability of a horse to pace). In one embodiment, the presence of the at least one biomarker is associated with a horse's ability to pace. In one embodiment of the invention, the method involves obtaining a physiological sample from a horse, wherein the sample comprises nucleic acid, and determining the presence of the biomarker.

The term “biomarker” is generally defined herein as a biological indicator, such as a particular molecular feature, that may affect or be related to diagnosing or predicting a subject's health or trait. For example, in certain embodiments of the present invention, the biomarker comprises a single nucleotide polymorphism (SNP) described herein, e.g., a particular genotype at a SNP described herein.

In one embodiment of the method, the at least one biomarker is a SNP described in table 3 or Table 4. In one embodiment of the method, the at least one biomarker is a SNP located at nucleotide position 14640812 on equine (ECA) chromosome 23, a SNP located at nucleotide position 28347510 on ECA17, a SNP located at nucleotide position 14107178 on ECA30, a SNP located at nucleotide position 14067984 on ECA30, a SNP located at nucleotide position 17945265 on ECA1, a SNP located at nucleotide position 81651604 on ECA6 and/or a SNP located at nucleotide position 15044553 on ECA25. In one embodiment of the method, the at least one biomarker is a SNP located at nucleotide position 14640812 on equine (ECA) chromosome 23. In one embodiment of the method, the at least one biomarker is a SNP located at nucleotide position 28347510 on ECA17. In one embodiment of the method, the at least one biomarker is a SNP located at nucleotide position 14107178 on ECA30. In one embodiment of the method, the at least one biomarker is a SNP located at nucleotide position 14067984 on ECA30. In one embodiment of the method, the at least one biomarker is a SNP located at nucleotide position 17945265 on ECA1. In one embodiment of the method, the at least one biomarker is a SNP located at nucleotide position 81651604 on ECA6. In one embodiment of the method, the at least one biomarker is a SNP located at nucleotide position 15044553 on ECA25.

In certain embodiments, the at least one biomarker is a variant (e.g., a SNP, a deletion, an insertion, etc.) that is in linkage disequilibrium with a SNP described herein (e.g., in Table 3 or 4). In certain embodiments, the at least one biomarker is a variant (e.g., a SNP, a deletion, an insertion, etc.) that is in linkage disequilibrium with a SNP located at nucleotide position 14640812 on equine (ECA) chromosome 23, a SNP located at nucleotide position 28347510 on ECA17, a SNP located at nucleotide position 14107178 on ECA30, a SNP located at nucleotide position 14067984 on ECA30, a SNP located at nucleotide position 17945265 on ECA1, a SNP located at nucleotide position 81651604 on ECA6 or a SNP located at nucleotide position 15044553 on ECA25.

As used herein, nucleotide position numbers refer to EquCab2, September 2007 (Wade et al., “Genome sequence, comparative analysis, and population genetics of the domestic horse”, Science, 326(5954):865-7 (2009)). These positions may vary depending on the assembly. Accordingly, in certain embodiments, the nucleotide position of a given SNP described herein is at a corresponding or alignable position in a different assembly.

TABLE A
Sequences of Certain SNPs described herein.

| | Nucleotide | | |
|---|---|---|---|
| Chr | Position | Relative Location | Sequence |
| 23 | 14640812 | ~6kb upstream of | CTCACATCCAAATTTTGTGATATTGTGCTGGCTTGAGC |
| | | ENSECAG00000010004 | TCCAGGGGAGCTGGCTCTGGTAAGGAAGGCTTCAGAT |
| | | | GCCCTCATTGTGGTGCTAACTGCAAGAAGAGCCCCTT |
| | | | GAGCCCCTTGGATGCAGAGCTGCAGTGTTCCTGTGAG |
| | | | A[A/G]CCTGTAAGGGACAGACAAGTGCTCTGTGGGGG |
| | | | CGGGGAGTCGTAATGGGGAATATAAAACACACACACC |
| | | | TTGCATAAGGCCTTACATTTCATCAGTGAGTACTGTG |
| | | | GGCACATGTGTTATTGCAGAGAAACATCACTAGCTGG |
| | | | AGCACCT (SEQ ID NO: 1) |
| 17 | 28347510 | Located within exon | CTGCTATCATACCCATCTAAAGGGACTTTCAGCAAGT |
| | | 15 of VWA8 | TGAAACAAGACTTTTAACAGACTGTCTTTGCCTTTTGG |
| | | | TAGGCACAATCATTGGCAGCATCACTTTCAACCAGAC |
| | | | AGCTACTGCGGATTTCTCGTCGGCTCTCACAGTACCCT |
| | | | [A/G]ATGAAAATCTTCACAGTGCTGTTACTAAAGCCTG |
| | | | CCTTTCCAGGTAACCATATTCTCCTTTCTCACTGAATT |
| | | | CAAGTTTCTGGGTCTTTGCTGAAACAGTAACATAAGT |
| | | | TTAATTGATAGTCTGTACTCACAGGTCTGCAAAAAGT |
| | | | ATTG (SEQ ID NO: 2) |
| 30 | 14107178 | Intergenic | AGGTTTGCTACTAACACTGTCACATTTCCAGTTGATGA |
| | | | GAAAGGAAACAAGCAAAAAATATGCAGGGTCCCCTCT |
| | | | GTCCCCGCCTGCCCCTCCTCGATATGGTGATATGTATG |
| | | | TCAACTCCCGAACAGAATATTATGTTTCACCGTAGAT |
| | | | [C/A]AAAAACATCCCCATGGATCATTTTCTTTTCAAATC |
| | | | TAAAATGTAAAGAGTGAATGCTGACGGCTCTCTCCTT |
| | | | TATTTTCCTGTCCACCAACAAAAGGAATCATTGATAC |
| | | | AAAGGCCGCTCTGACTGAGGAGTTTAGTTTTCAGAAT |
| | | | CTAC (SEQ ID NO: 3) |
| 30 | 14067984 | 750 bp upstream of | TTCATCTTGGTCCCAGGATCCAGCCCCACCTTCTCCCT |
| | | RRP15 | CCCACCCGCGCGGTGCAGAGCGAGGGTGCAAAACCTT |
| | | | TGCTCTCGGCGACAGCCTCCTGCCTCCCTCTCGGGGCC |
| | | | AGCTTCTCCCCGAGGTCAGCCTCTGCCACCTGCTTTC |
| | | | [T/G]CAGCACTGCGTTCCCTTGAAGCAGCTACCTCAAT |
| | | | CTGCTTCTGTTTCCACCCCCACCGCGAACCTCCCCACC |
| | | | AACCCCACCCGCCAACTTGGGAGCAAGCTTCGTTCAC |
| | | | CATTCTACCTCCAAGGCTAGCAACAGGGCTGGCCTGT |
| | | | AGTG (SEQ ID NO: 4) |
| 1 | 17945265 | Located within intron | ATCTTTTAAAAAACACAAATCTGATCATGTCACCCTCT |
| | | 2 of NHLRC2 | TGCAGAAATCCCCTGTTCCTTGTATAAAAGCCAAATCT |
| | | | CACCTCACAGCACGTTCTCCACATCCTTCTTCCCCTCG |
| | | | CTCAGGGTGTTGCAGCAGCCTGGCGTTCCTTCAGCT |
| | | | [G/C]CTCAAGAGTATCATTATTCCTCTTGCTTTAAGGCCT |
| | | | TGACAGTTCCCTTGACCTGGAATGTTCTCATCCTTTCT |
| | | | CACCTAGTAAAGCCATACTTGTCTTTCAGATCTCAAGC |
| | | | TCAATTTTCACTCTTAAGAAAGCTTTCTTTAATTCCTC |
| | | | (SEQ ID NO: 5) |
| 6 | 81651604 | Located within an | CACATGACTGCCAGATGAGGGCAAGAGAACCCTTAAT |
| | | intron of LLPH | GGGTATGGGGTTTCCTCTTAGGGTGATGAAAATGTTT |
| | | | TGGAAATAGAGGTGATGGTCGGGCAACATCATGAATG |
| | | | TACTAAATGCCACTGAACTGTACACTTTACAGTGGTT |
| | | | AA[G/T]TTTATTTTATGTGAGTTTTAGCTCGATGGCAA |
| | | | GAAGAGCGAACATGCAGGGCTGGTGTGCCTGTGTGGA |
| | | | AACAGAGACAGCAGGGGTACAAAGTTTCAAGGAGGC |
| | | | CAGCTAAAACACAGCAGCAGGTCGAGAGACATGTGA |
| | | | GATGGAGGG (SEQ ID NO: 6) |
| 25 | 15044553 | Intergenic | CATTCCTTGGCTTCTGGCTGCGTGACTCCAATTTCTGC |
| | | | CTCTGTCTTCCCATGGCCTTCCCCTCTGGGTCTGTGTG |
| | | | TCACCAATCTTCCTCTCTTTTCTCTGATAAAGACACCA |
| | | | GTCGTTAGGTTTAGAGCCCACCCTAAATCTAGAATG |
| | | | [T/A]TCTCACTGCAAGACCCTTAACTTTATTACATCTGTA |
| | | | AAGACCCTATTTCCAAATAAGGTCATATTAGCAGGTA |
| | | | CTGAGGATTGGGATTTGCACCTATCTTTTGGGGACTA |
| | | | TTAAAGCCACTATTGTTGTCATTATTATTATTTTCAGT |
| | | | TC (SEQ ID NO: 7) |

These seven SNPs are described in Table 4 in Example 1, along with the alternative allele frequencies in pacer and trotter populations. For four SNPs, the alternate allele was more common in pacers, and in three SNPs the alternate allele was more common in trotters. In either case, the group with the lower allele frequency included very few homozygotes.

Thus, in certain embodiments, the absence of a guanine (G) nucleotide at 14640812 on ECA23 in one or both alleles, the presence of a guanine (G) nucleotide at 28347510 on ECA17 in one or both alleles, the absence of an adenine (A) nucleotide at 14107178 on ECA30 in one or both alleles, the presence of a guanine (G) nucleotide at 14067984 on ECA30 in one or both alleles, the presence of a cytosine (C) nucleotide at 17945265 on ECA1 in one or both alleles, the absence of a thymine (T) nucleotide at 81651604 on ECA6 in one or both alleles and/or the presence of an adenine (A) nucleotide at 15044553 on ECA25 in one or both alleles is indicative of a horse that has the ability to pace.

In one embodiment, a panel of biomarkers are detected, wherein in the panel comprises two or more SNPs (e.g., 2, 3, 4, 5, 6, 7, 8, 9, 10, 11, 12, 13, 14, 15, 16, 17, 18, 19, 20, 21, 22 or 23 or more SNPs, such as SNPs described herein (e.g., in Tables 3 or 4)), and wherein the genotypes of the SNPs are detected. In certain embodiments, the panel comprises 3 or more SNPs (e.g., SNPs described herein). In certain embodiments, the panel comprises 4 or more SNPs (e.g., SNPs described herein). In certain embodiments, the panel comprises 5 or more SNPs (e.g., SNPs described herein). In certain embodiments, the panel comprises 6 or more SNPs (e.g., SNPs described herein). In certain embodiments, the panel comprises 7 or more SNPs (e.g., SNPs described herein). In certain embodiments, the panel comprises 7 SNPs (e.g., SNPs described herein). In certain embodiments, the panel comprises at least 7 SNPs, wherein the SNPs are located at nucleotide position 14640812 on equine (ECA) chromosome 23, nucleotide position 28347510 on ECA17, nucleotide position 14107178 on ECA30, nucleotide position 14067984 on ECA30, nucleotide position 17945265 on ECA1, nucleotide position 81651604 on ECA6 and nucleotide position 15044553 on ECA25.

Certain embodiments of the invention also provide a method for identifying a horse having the ability to pace, comprising:

1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25; and

2) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

In certain embodiments, a method described herein further comprises obtaining or having obtained a physiological sample from the horse, wherein the sample comprises nucleic acid.

Certain embodiments of the invention also provide a method, comprising:

1) obtaining or having obtained a physiological sample from a horse, wherein the physiological sample comprises nucleic acid;

2) assaying or having assayed the sample to detect the genotype of at least one single nucleotide polymorphism (SNP) at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25; and

3) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

In certain embodiments, a method further comprises selecting and training the identified horse for racing.

In certain embodiments, a method further comprises selecting and breeding the identified horse.

In certain embodiments, a method further comprises obtaining a sperm or egg sample from the identified horse, wherein the sperm or egg sample is used in a breeding program.

Certain embodiments of the invention provide a method of detecting at least one genetic variation in a horse, comprising:

1) obtaining or having obtained a physiological sample from the horse, wherein the physiological sample comprises nucleic acid; and

2) assaying sample to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25.

Certain embodiments of the invention provide a method of measuring a panel of single nucleotide polymorphisms (SNPs) in a horse to predict whether the horse has the ability to pace, comprising:

1) obtaining or having obtained a physiological sample from the horse, wherein the physiological sample comprises nucleic acid; and

2) assaying sample to detect the genotype of each SNP in the panel, wherein the panel comprises two or more SNPs selected from the group consisting of a SNP located at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25.

In certain embodiments, the genotypes of a panel of SNPs are detected, and wherein the panel comprises two or more SNPs (e.g., 2, 3, 4, 5, 6, 7, 8, 9, 10, 11, 12, 13, 14, 15, 16, 17, 18, 19, 20, 21, 22, 23 or more SNPs (e.g., SNPs described herein, such as in Table 3 or 4)). In certain embodiments, the panel comprises 3 or more SNPs (e.g., SNPs described herein). In certain embodiments, the panel comprises 4 or more SNPs (e.g., SNPs described herein). In certain embodiments, the panel comprises 5 or more SNPs (e.g., SNPs described herein). In certain embodiments, the panel comprises 6 or more SNPs (e.g., SNPs described herein). In certain embodiments, the panel comprises 7 or more SNPs (e.g., SNPs described herein). In certain embodiments, the panel comprises 7 SNPs (e.g., SNPs described herein). In certain embodiments, the panel comprises at least 7 SNPs, wherein the SNPs are located at nucleotide position 14640812 on equine (ECA) chromosome 23, nucleotide position 28347510 on ECA17, nucleotide position 14107178 on ECA30, nucleotide position 14067984 on ECA30, nucleotide position 17945265 on ECA1, nucleotide position 81651604 on ECA6 and nucleotide position 15044553 on ECA25.

In certain embodiments, a method described herein further comprises identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

In certain embodiments, a method described herein further comprises identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

In certain embodiments, a method described herein further comprises selecting the horse for training or breeding when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

In certain embodiments, a method described herein further comprises selecting the horse for training or breeding when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

In certain embodiments of the invention, the absence of a guanine (G) nucleotide at 14640812 on ECA23 in one or both alleles is detected. In certain embodiments of the invention, the absence of a guanine (G) nucleotide at 14640812 on ECA23 in one allele is detected. In certain embodiments of the invention, the absence of a guanine (G) nucleotide at 14640812 on ECA23 in both alleles is detected.

In certain embodiments of the invention, the presence of a guanine (G) nucleotide at 28347510 on ECA17 in one or both alleles is detected. In certain embodiments of the invention, the presence of a guanine (G) nucleotide at 28347510 on ECA17 in one allele is detected. In certain embodiments of the invention, the presence of a guanine (G) nucleotide at 28347510 on ECA17 in both alleles is detected.

In certain embodiments of the invention, the absence of an adenine (A) nucleotide at 14107178 on ECA30 in one or both alleles is detected. In certain embodiments of the invention, the absence of an adenine (A) nucleotide at 14107178 on ECA30 in one allele is detected. In certain embodiments of the invention, the absence of an adenine (A) nucleotide at 14107178 on ECA30 in both alleles is detected.

In certain embodiments of the invention, the presence of a guanine (G) nucleotide at 14067984 on ECA30 in one or both alleles is detected. In certain embodiments of the invention, the presence of a guanine (G) nucleotide at 14067984 on ECA30 in one allele is detected. In certain embodiments of the invention, the presence of a guanine (G) nucleotide at 14067984 on ECA30 in both alleles is detected.

In certain embodiments of the invention, the presence of a cytosine (C) nucleotide at 17945265 on ECA1 in one or both alleles is detected. In certain embodiments of the invention, the presence of a cytosine (C) nucleotide at 17945265 on ECA1 in one allele is detected. In certain embodiments of the invention, the presence of a cytosine (C) nucleotide at 17945265 on ECA1 in both alleles is detected.

In certain embodiments of the invention, the absence of a thymine (T) nucleotide at 81651604 on ECA6 in one or both alleles is detected. In certain embodiments of the invention, the absence of a thymine (T) nucleotide at 81651604 on ECA6 in one allele is detected. In certain embodiments of the invention, the absence of a thymine (T) nucleotide at 81651604 on ECA6 in both alleles is detected.

In certain embodiments of the invention, the presence of an adenine (A) nucleotide at 15044553 on ECA25 in one or both alleles is detected. In certain embodiments of the invention, the presence of an adenine (A) nucleotide at 15044553 on ECA25 in one allele is detected. In certain embodiments of the invention, the presence of an adenine (A) nucleotide at 15044553 on ECA25 in both alleles is detected.

In certain embodiments of the invention, the absence of a guanine (G) nucleotide at 14947553 on ECA30 in one or both alleles is detected. In certain embodiments of the invention, the absence of a guanine (G) nucleotide at 14947553 on ECA30 in one allele is detected. In certain embodiments of the invention, the absence of a guanine (G) nucleotide at 14947553 on ECA30 in both alleles is detected.

In certain embodiments of the invention, the presence of a guanine (G) nucleotide at 28361747 on ECA17 in one or both alleles is detected. In certain embodiments of the invention, the presence of a guanine (G) nucleotide at 28361747 on ECA17 in one allele is detected. In certain embodiments of the invention, the presence of a guanine (G) nucleotide at 28361747 on ECA17 in both alleles is detected.

In certain embodiments of the invention, the presence of an adenine (A) nucleotide at 14648590 on ECA23 in one or both alleles is detected. In certain embodiments of the invention, the presence of an adenine (A) nucleotide at 14648590 on ECA23 in one allele is detected. In certain embodiments of the invention, the presence of an adenine (A) nucleotide at 14648590 on ECA23 in both alleles is detected.

In certain embodiments of the invention, the absence of a cytosine (C) nucleotide at 14649864 on ECA23 in one or both alleles is detected. In certain embodiments of the invention, the absence of a cytosine (C) nucleotide at 14649864 on ECA23 in one allele is detected. In certain embodiments of the invention, the absence of a cytosine (C) nucleotide at 14649864 on ECA23 in both alleles is detected.

In certain embodiments of the invention, the presence of a guanine (G) nucleotide at 15068782 on ECA30 in one or both alleles is detected. In certain embodiments of the invention, the presence of a guanine (G) nucleotide at 15068782 on ECA30 in one allele is detected. In certain embodiments of the invention, the presence of a guanine (G) nucleotide at 15068782 on ECA30 in both alleles is detected.

In certain embodiments of the invention, the presence of a thymine (T) nucleotide at 35731283 on ECA1 in one or both alleles is detected. In certain embodiments of the invention, the presence of a thymine (T) nucleotide at 35731283 on ECA1 in one allele is detected. In certain embodiments of the invention, the presence of a thymine (T) nucleotide at 35731283 on ECA1 in both alleles is detected.

In certain embodiments of the invention, the presence of an adenine (A) nucleotide at 20652865 on ECA23 in one or both alleles is detected. In certain embodiments of the invention, the presence of an adenine (A) nucleotide at 20652865 on ECA23 in one allele is detected. In certain embodiments of the invention, the presence of an adenine (A) nucleotide at 20652865 on ECA23 in both alleles is detected.

In certain embodiments of the invention, the presence of an adenine (A) nucleotide at 28540291 on ECA17 in one or both alleles is detected. In certain embodiments of the invention, the presence of an adenine (A) nucleotide at 28540291 on ECA17 in one allele is detected. In certain embodiments of the invention, the presence of an adenine (A) nucleotide at 28540291 on ECA17 in both alleles is detected.

In certain embodiments of the invention, the presence of a cytosine (C) nucleotide at 15055793 on ECA30 in one or both alleles is detected. In certain embodiments of the invention, the presence of a cytosine (C) nucleotide at 15055793 on ECA30 in one allele is detected. In certain embodiments of the invention, the presence of a cytosine (C) nucleotide at 15055793 on ECA30 in both alleles is detected.

In certain embodiments of the invention, the presence of a guanine (G) nucleotide at 35731849 on ECA1 in one or both alleles is detected. In certain embodiments of the invention, the presence of a guanine (G) nucleotide at 35731849 on ECA1 in one allele is detected. In certain embodiments of the invention, the presence of a guanine (G) nucleotide at 35731849 on ECA1 in both alleles is detected.

In certain embodiments of the invention, the presence of a thymine (T) nucleotide at 35729338 on ECA1 in one or both alleles is detected. In certain embodiments of the invention, the presence of a thymine (T) nucleotide at 35729338 on ECA1 in one allele is detected. In certain embodiments of the invention, the presence of a thymine (T) nucleotide at 35729338 on ECA1 in both alleles is detected.

In certain embodiments of the invention, the presence of an adenine (A) nucleotide at 14936139 on ECA30 in one or both alleles is detected. In certain embodiments of the invention, the presence of an adenine (A) nucleotide at 14936139 on ECA30 in one allele is detected. In certain embodiments of the invention, the presence of an adenine (A) nucleotide at 14936139 on ECA30 in both alleles is detected.

In certain embodiments of the invention, the presence of an adenine (A) nucleotide at 20662320 on ECA23 in one or both alleles is detected. In certain embodiments of the invention, the presence of an adenine (A) nucleotide at 20662320 on ECA23 in one allele is detected. In certain embodiments of the invention, the presence of an adenine (A) nucleotide at 20662320 on ECA23 in both alleles is detected.

In certain embodiments of the invention, the presence of a cytosine (C) nucleotide at 35720250 on ECA1 in one or both alleles is detected. In certain embodiments of the invention, the presence of a cytosine (C) nucleotide at 35720250 on ECA1 in one allele is detected. In certain embodiments of the invention, the presence of a cytosine (C) nucleotide at 35720250 on ECA1 in both alleles is detected.

In certain embodiments of the invention, the presence of a cytosine (C) nucleotide at 35721326 on ECA1 in one or both alleles is detected. In certain embodiments of the invention, the presence of a cytosine (C) nucleotide at 35721326 on ECA1 in one allele is detected. In certain embodiments of the invention, the presence of a cytosine (C) nucleotide at 35721326 on ECA1 in both alleles is detected.

In certain embodiments of the invention, the presence of a thymine (T) nucleotide at 35726345 on ECA1 in one or both alleles is detected. In certain embodiments of the invention, the presence of a thymine (T) nucleotide at 35726345 on ECA1 in one allele is detected. In certain embodiments of the invention, the presence of a thymine (T) nucleotide at 35726345 on ECA1 in both alleles is detected.

In certain embodiments, the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and the presence of a thymine (T) nucleotide at 35726345 on ECA1 in one or both alleles is detected.

In certain embodiments of the invention, the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and the presence of an adenine (A) nucleotide at 15044553 on ECA25 in one or both alleles is detected.

In certain embodiments, the absence of a guanine (G) nucleotide at 14640812 on ECA23 in both alleles is detected, the presence of a guanine (G) nucleotide at 28347510 on ECA17 in one allele is detected, the absence of an adenine (A) nucleotide at 14107178 on ECA30 in one allele is detected, the presence of a guanine (G) nucleotide at 14067984 on ECA30 in one allele is detected, the presence of a cytosine (C) nucleotide at 17945265 on ECA1 in one allele is detected, the absence of a thymine (T) nucleotide at 81651604 on ECA6 in one allele is detected, the presence of an adenine (A) nucleotide at 15044553 on ECA25 in one allele is detected, the absence of a guanine (G) nucleotide at 14947553 on ECA30 in one allele is detected, the presence of a guanine (G) nucleotide at 28361747 on ECA17 in one allele is detected, the presence of an adenine (A) nucleotide at 14648590 on ECA23 in one allele is detected, the absence of a cytosine (C) nucleotide at 14649864 on ECA23 in one allele is detected, the presence of a guanine (G) nucleotide at 15068782 on ECA30 in one allele is detected, the presence of a thymine (T) nucleotide at 35731283 on ECA1 in one allele is detected, the presence of an adenine (A) nucleotide at 20652865 on ECA23 in one allele is detected, the presence of an adenine (A) nucleotide at 28540291 on ECA17 in one allele is detected, the presence of a cytosine (C) nucleotide at 15055793 on ECA30 in one allele is detected, the presence of a guanine (G) nucleotide at 35731849 on ECA1 in one allele is detected, the presence of a thymine (T) nucleotide at 35729338 on ECA1 in one allele is detected, the presence of an adenine (A) nucleotide at 14936139 on ECA30 in one allele is detected, the presence of an adenine (A) nucleotide at 20662320 on ECA23 in one allele is detected, the presence of a cytosine (C) nucleotide at 35720250 on ECA1 in one allele is detected, the presence of a cytosine (C) nucleotide at 35721326 on ECA1 in one allele is detected and the presence of a thymine (T) nucleotide at 35726345 on ECA1 in one allele is detected.

In certain embodiments of the invention, the absence of a guanine (G) nucleotide at 14640812 on ECA23 in one allele is detected, the presence of a guanine (G) nucleotide at 28347510 on ECA17 in one allele is detected, the absence of an adenine (A) nucleotide at 14107178 on ECA30 in one allele is detected, the presence of a guanine (G) nucleotide at 14067984 on ECA30 in one allele is detected, the presence of a cytosine (C) nucleotide at 17945265 on ECA1 in one allele is detected, the absence of a thymine (T) nucleotide at 81651604 on ECA6 in one allele is detected and the presence of an adenine (A) nucleotide at 15044553 on ECA25 in one allele is detected.

In certain embodiments, the absence of a guanine (G) nucleotide at 14640812 on ECA23 in both alleles is detected, the presence of a guanine (G) nucleotide at 28347510 on ECA17 in both alleles is detected, the absence of an adenine (A) nucleotide at 14107178 on ECA30 in both alleles is detected, the presence of a guanine (G) nucleotide at 14067984 on ECA30 in both alleles is detected, the presence of a cytosine (C) nucleotide at 17945265 on ECA1 in both alleles is detected, the absence of a thymine (T) nucleotide at 81651604 on ECA6 in both alleles is detected, the presence of an adenine (A) nucleotide at 15044553 on ECA25 in both alleles is detected, the absence of a guanine (G) nucleotide at 14947553 on ECA30 in both alleles is detected, the presence of a guanine (G) nucleotide at 28361747 on ECA17 in both alleles is detected, the presence of an adenine (A) nucleotide at 14648590 on ECA23 in both alleles is detected, the absence of a cytosine (C) nucleotide at 14649864 on ECA23 in both alleles is detected, the presence of a guanine (G) nucleotide at 15068782 on ECA30 in both alleles is detected, the presence of a thymine (T) nucleotide at 35731283 on ECA1 in both alleles is detected, the presence of an adenine (A) nucleotide at 20652865 on ECA23 in both alleles is detected, the presence of an adenine (A) nucleotide at 28540291 on ECA17 in both alleles is detected, the presence of a cytosine (C) nucleotide at 15055793 on ECA30 in both alleles is detected, the presence of a guanine (G) nucleotide at 35731849 on ECA1 in both alleles is detected, the presence of a thymine (T) nucleotide at 35729338 on ECA1 in both alleles is detected, the presence of an adenine (A) nucleotide at 14936139 on ECA30 in both alleles is detected, the presence of an adenine (A) nucleotide at 20662320 on ECA23 in both alleles is detected, the presence of a cytosine (C) nucleotide at 35720250 on ECA1 in both alleles is detected, the presence of a cytosine (C) nucleotide at 35721326 on ECA1 in both alleles is detected and the presence of a thymine (T) nucleotide at 35726345 on ECA1 in both alleles is detected.

In certain embodiments of the invention, the absence of a guanine (G) nucleotide at 14640812 on ECA23 in both alleles is detected, the presence of a guanine (G) nucleotide at 28347510 on ECA17 in both alleles is detected, the absence of an adenine (A) nucleotide at 14107178 on ECA30 in both alleles is detected, the presence of a guanine (G) nucleotide at 14067984 on ECA30 in both alleles is detected, the presence of a cytosine (C) nucleotide at 17945265 on ECA1 in both alleles is detected, the absence of a thymine (T) nucleotide at 81651604 on ECA6 in both alleles is detected and the presence of an adenine (A) nucleotide at 15044553 on ECA25 in both alleles is detected.

In certain embodiments, a method described herein further comprises (e.g., the identifying step further comprises) using a learning statistical classifier system to analyze the SNP genotypes. Learning statistical classifier systems are known in the art, and include, but are not limited to, e.g., Random Forest (RF), Classification and Regression Tree (CART), boosted tree, neural network (NN), support vector machine (SVM), general chi-squared automatic interaction detector model, interactive tree, multiadpative regression spline, machine learning classifier, and combinations thereof.

In certain embodiments, the learning statistical classifier system is a Random Forest system. In certain embodiments, the Random Forest system is used as described in the Example.

In certain embodiments, the panel has a misclassification error of less than about 20%. In certain embodiments, the panel has a misclassification error of less than about 18%. In certain embodiments, the panel has a misclassification error of less than about 15%. In certain embodiments, the panel has a misclassification error of less than about 12%. In certain embodiments, the panel has a misclassification error of less than about 10%. In certain embodiments, the panel has a misclassification error of less than about 8%. In certain embodiments, the panel has a misclassification error of less than about 6%. In certain embodiments, the panel has a misclassification error of less than about 5%. In certain embodiments, the panel has a misclassification error of less than about 4%. In certain embodiments, the panel has a misclassification error of less than about 3%. In certain embodiments, the panel has a misclassification error of less than about 2%. In certain embodiments, the panel has a misclassification error of less than about 1%.

As discussed above, the methods of the present invention can be used to detect the presence of at least one biomarker associated with an alternative gait (e.g., a pace gait) in a horse such as a foal, e.g., one of a breeding pair of horses, e.g., the potential dam and/or sire, or any horse at any stage of life. Horse breeds may be classified as gaited or non-gaited, wherein gaited horses have the ability to perform alternative gaits in addition to the three basic gaits (i.e., walk, trot and gallop). Examples of gaited breeds include, but are not limited to, American Saddlebred, Campolina, Icelandic horse, Kentucky Mountain Saddle Horse, Mangalarga Marchador, Marwari horse, Missouri Foxtrotter, Paso Fino, Racking horse, Rocky Mountain horse, Spotted Saddle horse, Standardbred, Tennessee Walker and Walkaloosa. Examples of non-gaited breeds include, but are not limited to, Akhal Teke, American Paint Horse, Andalusian, Arabian, Belgian, Dole, Exmoor Pony, Friesian, Haflinger, Hanoverian, Lusitano, North Swedish Draft horse, Norwegian Fjord, Quarter Horse, Selle Francais, Shetland pony, Suffolk Punch, Thoroughbred and Trakehner. In certain embodiments, the horse is a gaited horse. In certain embodiments, the horse is a gaited horse selected from the group consisting of American Saddlebred, Campolina, Icelandic horse, Kentucky Mountain Saddle Horse, Mangalarga Marchador, Marwari horse, Missouri Foxtrotter, Paso Fino, Racking horse, Rocky Mountain horse, Spotted Saddle horse, Standardbred, Tennessee Walker and Walkaloosa. In certain embodiments, the horse is a Standardbred.

Further provided by the present invention is a kit comprising a diagnostic test for identifying a horse that has the ability to pace comprising packaging material, containing, separately packaged, at least one oligonucleotide probe capable of forming a hybridized nucleic acid with a SNP described herein or a nucleic acid region flanking a SNP described herein and instructions directing the use of the probe in accordance with the methods of the invention. In certain embodiments, the oligonucleotide probe is a first primer that hybridizes 5′ or 3′ to a SNP described herein.

In certain embodiments, the least one oligonucleotide probe capable of forming a hybridized nucleic acid with a SNP or a nucleic acid region flanking a SNP, wherein the SNP is selected from the group consisting of a SNP at 14640812 on ECA23, a SNP at 28347510 on ECA17, a SNP at 14107178 on ECA30, a SNP at 14067984 on ECA30, a SNP at 17945265 on ECA1, a SNP at 81651604 at ECA6, a SNP at 15044553 on ECA25, a SNP at 14947553 on ECA30, a SNP at 28361747 on ECA17, a SNP at 14648590 on ECA23, a SNP at 14649864 on ECA23, a SNP at 15068782 on ECA30, a SNP at 35731283 on ECA1, a SNP at 20652865 on ECA23, a SNP at 28540291 on ECA17, a SNP at 15055793 on ECA30, a SNP at 35731849 on ECA1, a SNP at 35729338 on ECA1, a SNP at 14936139 on ECA30, a SNP at 20662320 on ECA23, a SNP at 35720250 on ECA1, a SNP at 35721326 on ECA1 and a SNP at 35726345 on ECA1. In certain embodiments, the SNP is selected from the group consisting of a SNP at 14640812 on ECA23, a SNP at 28347510 on ECA17, a SNP at 14107178 on ECA30, a SNP at 14067984 on ECA30, a SNP at 17945265 on ECA1, a SNP at 81651604 at ECA6 and a SNP at 15044553 on ECA25.

In certain embodiments, the kit further comprises a second oligonucleotide probe capable of forming a hybridized nucleic acid with a SNP described herein or a nucleic acid region flanking a SNP described herein. In certain embodiments, the second oligonucleotide probe is a second primer that hybridizes 5′ or 3′ to a SNP described herein. In certain embodiments, the first and second primers are capable of amplifying a SNP described herein (i.e., the first and second primers are a primer pair). In certain embodiments, the first primer and the second primer hybridize to a region in the range of between about 50 to about 1000 base pairs, or about 50 to about 500 base pairs, or to about 50 to about 400 base pairs, or to about 50 to about 300 base pairs, or to about 50 to about 200 base pairs, or to about 50 to about 100 base pairs.

In certain embodiments, the kit comprises primer pairs capable of amplifying SNPs (e.g., described herein). In certain embodiments, the kits comprises primers for amplifying at least 2, 3, 4, 5, 6, 7, 8, 9, 10, 11, 12, 13, 14, 15, 16, 17, 18, 19, 20, 21, 22, 23 or more SNPs (e.g., described herein).

### Nucleic Acids of the Invention

Sources of nucleotide sequences from which the present nucleic acid molecules can be obtained include any prokaryotic or eukaryotic source. For example, they can be obtained from a mammalian, such as an equine, cellular source. Alternatively, nucleic acid molecules of the present invention can be obtained from a library, such as the library described in McCoy et al., BMC Genomics. 2016; 17:41. doi: 10.1186/s12864-016-2385-z.

DNA extraction, isolation and purification methods are well-known in the art and can be applied in the present invention. Standard protocols for the isolation of genomic DNA are inter alia referred to in Sambrook, J., Russell, D. W. Molecular Cloning: A Laboratory Manual, the third edition, Cold Spring Harbor Laboratory Press, Cold Spring Harbor. New York, 1.31-1.38, 2001 and Sharma. R. C., et al. “A rapid procedure for isolation of RNA-free genomic DNA from mammalian cells”, BioTechniques, 14. 176-178. 1993.

Certain embodiments of the invention provide a nucleic acid described herein (e.g., isolated), or a portion thereof. In certain embodiments, the nucleic acid comprises a SNP described herein (e.g., as described in Table 3 or Table 4). In certain embodiments, the nucleic acid comprises a SNP described herein (e.g., as described in Table 3 or Table 4), other than the SNP present at 81651604 on Chr 6. In certain embodiments, the nucleic acid comprises a SNP at 14640812 on ECA23. In certain embodiments, the nucleic acid comprises a SNP at 28347510 on ECA17. In certain embodiments, the nucleic acid comprises a SNP at 14107178 on ECA30. In certain embodiments, the nucleic acid comprises a SNP at 14067984 on ECA30. In certain embodiments, the nucleic acid comprises a SNP at 17945265 on ECA1. In certain embodiments, the nucleic acid comprises a SNP at 15044553 on ECA25. In certain embodiments, the nucleic acid comprises a SNP at 14947553 on ECA30. In certain embodiments, the nucleic acid comprises a SNP at 28361747 on ECA17. In certain embodiments, the nucleic acid comprises a SNP at 14648590 on ECA23. In certain embodiments, the nucleic acid comprises a SNP at 14649864 on ECA23. In certain embodiments, the nucleic acid comprises a SNP at 15068782 on ECA30. In certain embodiments, the nucleic acid comprises a SNP at 35731283 on ECA1. In certain embodiments, the nucleic acid comprises a SNP at 20652865 on ECA23. In certain embodiments, the nucleic acid comprises a SNP at 28540291 on ECA17. In certain embodiments, the nucleic acid comprises a SNP at 15055793 on ECA30. In certain embodiments, the nucleic acid comprises a SNP at 35731849 on ECA1. In certain embodiments, the nucleic acid comprises a SNP at 35729338 on ECA1. In certain embodiments, the nucleic acid comprises a SNP at 14936139 on ECA30. In certain embodiments, the nucleic acid comprises a SNP at 20662320 on ECA23. In certain embodiments, the nucleic acid comprises a SNP at 35720250 on ECA1. In certain embodiments, the nucleic acid comprises a SNP at 35721326 on ECA1. In certain embodiments, the nucleic acid comprises a SNP at 35726345 on ECA1.

In certain embodiments, the nucleic acid is about 5 to about 2000 base pairs in length, about 5 to about 1750 base pairs in length, about 5 to about 1500 base pairs in length, about 5 to about 1250 base pairs in length, about 5 to about 1000 base pairs in length, about 5 to about 900 base pairs in length, about 5 to about 800 base pairs in length, about 5 to about 700 base pairs in length, about 5 to about 600 base pairs in length, about 5 to about 500 base pairs in length, about 5 to about 400 base pairs in length, about 5 to about 300 base pairs in length, about 5 to about 200 base pairs in length, about 5 to about 175 base pairs in length, about 5 to about 150 base pairs in length, about 5 to about 125 base pairs in length, about 5 to about 100 base pairs in length, about 5 to about 75 base pairs in length, about 5 to about 50 base pairs in length, about 10 to about 50 base pairs in length, about 10 to about 40 base pairs in length or about 10 to about 30 base pairs in length.

In certain embodiments, the nucleic acid comprises a nucleotide sequence having at least about 80%, 85%, 90%, 95%, 96%, 97%, 98%, 99% or 100% sequence identity to SEQ ID NO:1, SEQ ID NO:2, SEQ ID NO:3, SEQ ID NO:4, SEQ ID NO:5, SEQ ID NO:6 or SEQ ID NO:7, or a portion thereof. In certain embodiments, the nucleic acid comprises a nucleotide sequence having at least about 80%, 85%, 90%, 95%, 96%, 97%, 98%, 99% or 100% sequence identity to SEQ ID NO:1, SEQ ID NO:2, SEQ ID NO:3, SEQ ID NO:4, SEQ ID NO:5 or SEQ ID NO:7, or a portion thereof.

In certain embodiments, the nucleic acid further comprises a promoter.

Certain embodiments of the invention provide an expression cassette comprising a nucleic acid sequence described herein and a promoter operably linked to the nucleic acid.

In certain embodiments, the promoter is a regulatable promoter. In certain embodiments, the promoter is a constitutive promoter.

In certain embodiments, the expression cassette further comprises an expression control sequence (e.g., an enhancer) operably linked to the nucleic acid sequence. Expression control sequences and techniques for operably linking sequences together are well known in the art.

Certain embodiments of the invention provide a vector comprising an expression cassette described herein.

Certain embodiments of the invention provide a polypeptide comprising an amino acid sequence encoded by a nucleic acid described herein.

Certain embodiments of the invention provide a cell comprising a nucleic acid described herein, an expression cassette described herein or a vector described herein. Certain embodiments of the invention provide a cell comprising a polypeptide described herein.

As discussed above, the terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule that is complementary or hybridizes to a sequence of interest, i.e., a nucleic acid sequence comprising a SNP described herein (e.g., Table 3 or Table 4), and remains stably bound under stringent conditions (as defined by methods well known in the art). Thus, the RNA or DNA is “isolated” in that it is free from at least one contaminating nucleic acid with which it is normally associated in the natural source of the RNA or DNA and in one embodiment of the invention is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

### Oligonucleotide Probes and Hybridization Products

As noted above, the method of the present invention is useful for detecting the presence of a polymorphism in equine DNA, in particular, the presence of particular alleles at a given SNP described herein (e.g., Table 3 or Table 4).

A single primer or primer pairs may be useful for determination of the nucleotide sequence of a particular SNP using PCR. For example, pairs of single-stranded DNA primers can be annealed to sequences comprising or surrounding the SNP in order to prime amplifying DNA synthesis of the SNP itself. Allele-specific primers can also be used. Such primers anneal only to particular alleles, and thus will only amplify a product in the presence of the particular variant allele as a template. Accordingly, certain embodiments described herein, provide a primer capable of hybridizing to a nucleic acid sequence comprising or surrounding a SNP described herein. Certain embodiments also provide a primer pair capable of amplifying a nucleic acid sequence comprising or surrounding a SNP described herein. Certain embodiments provide a primer or primer pair useful for determining the genotype of a SNP described herein.

In certain embodiments, an assay described herein comprises contacting the sample with at least one oligonucleotide probe to form at least one hybridized nucleic acid product. The oligonucleotide probes that are useful in the methods of the present invention can be any probe comprised of between about 4 or 6 bases up to about 80 or 100 bases or more. In one embodiment of the present invention, the probes are between about 10 and about 20 bases.

The primers themselves can be synthesized using techniques that are well known in the art. Generally, the primers can be made using oligonucleotide synthesizing machines that are commercially available. Given the sequences flanking the SNPs described herein are provided above/known in the art, design of particular primers is well within the skill of the art.

Oligonucleotide probes may be prepared having any of a wide variety of base sequences according to techniques that are well known in the art. Suitable bases for preparing the oligonucleotide probe may be selected from naturally occurring nucleotide bases such as adenine, cytosine, guanine, uracil, and thymine; and non-naturally occurring or “synthetic” nucleotide bases such as 7-deaza-guanine 8-oxo-guanine, 6-mercaptoguanine, 4-acetylcytidine, 5-(carboxyhydroxymethyl)uridine, 2′-O-methylcytidine, 5-carboxymethylamino-methyl-2-thioridine, 5-carboxymethylaminomethyluridine, dihydrouridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylamninomethyluridine, 5-methoxyaminomethyl-2-thiouridine, β,D-mannosylqueosine, 5-methloxycarbonylmethyluridine, 5-methoxyuridine, 2-methyltio-N6-isopentenyladenosine, N-((9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-Methylurdine, N-((9-beta-D-ribofuranosylpurine-6-yl)carbamoyl)threonine, 2′-O-methyl-5-methyluridine, 2′-O-methylurdine, wybutosine, and 3-(3-amino-3-carboxypropyl)uridine. Any oligonucleotide backbone may be employed, including DNA, RNA (although RNA is less preferred than DNA), modified sugars such as carbocycles, and sugars containing 2′ substitutions such as fluoro and methoxy. The oligonucleotides may be oligonucleotides wherein at least one, or all, of the internucleotide bridging phosphate residues are modified phosphates, such as methyl phosphonates, methyl phosphonotlioates, phosphoroinorpholidates, phosphoropiperazidates and phosplioramidates (for example, every other one of the internucleotide bridging phosphate residues may be modified as described). The oligonucleotide may be a “peptide nucleic acid” such as described in Nielsen et al., Science, 254, 1497-1500 (1991).

The only requirement is that the oligonucleotide probe should possess a sequence at least a portion of which is capable of binding to a known portion of the sequence of the DNA sample.

It may be desirable in some applications to contact the DNA sample with a number of oligonucleotide probes having different base sequences (e.g., where there are two or more target nucleic acids in the sample, or where a single target nucleic acid is hybridized to two or more probes in a “sandwich” assay).

The nucleic acid probes provided by the present invention are useful for a number of purposes. The probes can be used to detect PCR amplification products. They may also be used to detect particular allelic variants using other techniques.

The DNA (or nucleic acid) sample may be contacted with the oligonucleotide probe in any suitable manner known to those skilled in the art. For example, the DNA sample may be solubilized in solution, and contacted with the oligonucleotide probe by solubilizing the oligonucleotide probe in solution with the DNA sample under conditions that permit hybridization. Suitable conditions are well known to those skilled in the art. Alternatively, the DNA sample may be solubilized in solution with the oligonucleotide probe immobilized on a solid support, whereby the DNA sample may be contacted with the oligonucleotide probe by immersing the solid support having the oligonucleotide probe immobilized thereon in the solution containing the DNA sample.

As described herein, method of the invention may comprise detecting a hybridized nucleic acid product. Methods of detection are known in the art, for example, e.g., by polymerase chain reaction (PCR), allele specific hybridization, the 3′exonuclease assay (Taqman assay), fluorescent dye and quenching agent-based PCR assay, allele-specific restriction enzymes (RFLP-based techniques), direct sequencing, the oligonucleotide ligation assay (OLA), pyrosequencing, the invader assay, minisequencing, DHPLC-based techniques, single strand conformational polymorphism (SSCP), allele-specific PCR, denaturating gradient gel electrophoresis (DGGE), temperature gradient gel electrophoresis (TGGE), chemical mismatch cleavage (CMG), heteroduplex analysis based system, techniques based on mass spectroscopy, invasive cleavage assay, polymorphism ratio sequencing (PRS), microarrays, a rolling circle extension assay, HPLC-based techniques, extension based assays, ARMS (Amplification Refractory Mutation System), ALEX (Amplification Refractory Mutation Linear Extension), SBCE (Single base chain extension), molecular beacon assays, invader (Third wave technologies), ligase chain reaction assays, 5′-nuclease assay-based techniques, hybridization capillary array electrophoresis (GAE), or solid phase hybridization (dot blot, reverse dot blot, chips).

Thus, in certain embodiments, the method further comprising detecting the at least one hybridized nucleic acid product, wherein the detection is performed by polymerase chain reaction (PCR), allele specific hybridization, the 3′exonuclease assay (Taqman assay), fluorescent dye and quenching agent-based PCR assay, allele-specific restriction enzymes (RFLP-based techniques), direct sequencing, the oligonucleotide ligation assay (OLA), pyrosequencing, the invader assay, minisequencing, DHPLC-based techniques, single strand conformational polymorphism (SSCP), allele-specific PCR, denaturating gradient gel electrophoresis (DGGE), temperature gradient gel electrophoresis (TGGE), chemical mismatch cleavage (CMG), heteroduplex analysis based system, techniques based on mass spectroscopy, invasive cleavage assay, polymorphism ratio sequencing (PRS), microarrays, a rolling circle extension assay, HPLC-based techniques, extension based assays, ARMS (Amplification Refractory Mutation System), ALEX (Amplification Refractory Mutation Linear Extension), SBCE (Single base chain extension), molecular beacon assays, invader (Third wave technologies), ligase chain reaction assays, 5′-nuclease assay-based techniques, hybridization capillary array electrophoresis (GAE), or solid phase hybridization (dot blot, reverse dot blot, chips). These methods are well known and widely practiced in the art.

### Nucleic Acid Amplification Methods

In certain embodiments, an assay step/detection step may further comprise amplifying a DNA present in a physiological sample or derived from a physiological sample (e.g., a hybridized nucleic acid product). Such amplification may be carried out by any means known to the art. For example, “amplifying” may utilize methods such as polymerase chain reaction (PCR), ligation amplification (or ligase chain reaction, LCR), strand displacement amplification, nucleic acid sequence-based amplification, and amplification methods based on the use of Q-beta replicase. These methods are well known and widely practiced in the art. Reagents and hardware for conducting PCR are commercially available. For example, in certain embodiments of the present invention, a region flanking a SNP described herein, may be amplified by PCR (see, e.g., SEQ ID NOs:1-7 or a portion thereof). In another embodiment of the present invention, at least one oligonucleotide probe is immobilized on a solid surface.

In certain embodiments, the at least one amplified nucleic acid product comprises a SNP located at nucleotide position described herein (e.g., Table 3 or 4). In certain embodiments, the at least one amplified nucleic acid product comprises a SNP located at nucleotide position 14640812 on ECA23, nucleotide position 28347510 on ECA17, nucleotide position 14107178 on ECA30, nucleotide position 14067984 on ECA30, nucleotide position 17945265 on ECA1, nucleotide position 81651604 on ECA6 or nucleotide position 15044553 on ECA25.

Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3 SR”), the Qβ replicase system, nucleic acid sequence-based amplification (or “NASBA”), the repair chain reaction (or “RCR”), and boomerang DNA amplification (or “BDA”).

The bases incorporated into the amplification product may be natural or modified bases (modified before or after amplification), and the bases may be selected to optimize subsequent electrochemical detection steps.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques. Where the nucleic acid to be amplified is RNA, amplification may be carried out by initial conversion to DNA by reverse transcriptase in accordance with known techniques.

Strand displacement amplification (SDA) may be carried out in accordance with known techniques. For example, SDA may be carried out with a single amplification primer or a pair of amplification primers, with exponential amplification being achieved with the latter. In general, SDA amplification primers comprise, in the 5′ to 3′ direction, a flanking sequence (the DNA sequence of which is noncritical), a restriction site for the restriction enzyme employed in the reaction, and an oligonucleotide sequence (e.g., an oligonucleotide probe of the present invention) that hybridizes to the target sequence to be amplified and/or detected. The flanking sequence, which serves to facilitate binding of the restriction enzyme to the recognition site and provides a DNA polymerase priming site after the restriction site has been nicked, is about 15 to 20 nucleotides in length in one embodiment. The restriction site is functional in the SDA reaction. The oligonucleotide probe portion is about 13 to 15 nucleotides in length in one embodiment of the invention.

Ligase chain reaction (LCR) is also carried out in accordance with known techniques. In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

In one embodiment of the invention, a region surrounding a SNP described herein (e.g., Table 3 or Table 4) is amplified by PCR using primers based on the known sequence. The amplified regions are then sequenced using automated sequencers. In this manner, the genotype at a given SNP may be established.

Diagnostic techniques that are useful in the methods of the invention include, but are not limited to direct DNA sequencing, PFGE analysis, allele-specific oligonucleotide (ASO), dot blot analysis and denaturing gradient gel electrophoresis, and are well known to the artisan.

There are several methods that can be used to detect DNA sequence variation. Direct DNA sequencing (e.g., Next-generation sequencing), either manual sequencing or automated fluorescent sequencing can detect sequence variation. Another approach is the single-stranded conformation polymorphism assay (SSCA). This method does not detect all sequence changes, especially if the DNA fragment size is greater than 200 bp, but can be optimized to detect most DNA sequence variation. The reduced detection sensitivity is a disadvantage, but the increased throughput possible with SSCA makes it an attractive, viable alternative to direct sequencing for mutation detection on a research basis. The fragments that have shifted mobility on SSCA gels are then sequenced to determine the exact nature of the DNA sequence variation. Other approaches based on the detection of mismatches between the two complementary DNA strands include clamped denaturing gel electrophoresis (CDGE), heteroduplex analysis (HA) and chemical mismatch cleavage (CMC). Once a mutation is known, an allele specific detection approach such as allele specific oligonucleotide (ASO) hybridization can be utilized to rapidly screen large numbers of other samples for that same mutation. Such a technique can utilize probes which are labeled with gold nanoparticles to yield a visual color result.

Detection of SNPs may be accomplished by molecular cloning of the region flanking the SNP and sequencing the allele(s) at the locus using techniques well known in the art. Alternatively, the sequences can be amplified directly from a genomic DNA preparation from equine tissue, using known techniques. The DNA sequence of the amplified sequences can then be determined.

There are six well known methods for a more complete, yet still indirect, test for confirming the presence of a particular allele: 1) single stranded conformation analysis (SSCA); 2) denaturing gradient gel electrophoresis (DGGE); 3) RNase protection assays; 4) allele-specific oligonucleotides (ASOs); 5) the use of proteins which recognize nucleotide mismatches, such as the E. coli mutS protein; and 6) allele-specific PCR. For allele-specific PCR, primers are used which hybridize at their 3′ ends to a particular allele of a given SNP. If the particular allele is not present, an amplification product is not observed. Amplification Refractory Mutation System (ARMS) can also be used.

In the first three methods (SSCA, DGGE and RNase protection assay), a new electrophoretic band appears. SSCA detects a band that migrates differentially because the sequence change causes a difference in single-strand, intramolecular base pairing. RNase protection involves cleavage of the variant polynucleotide into two or more smaller fragments. DGGE detects differences in migration rates of variant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between variant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations/variations in the gene or in its mRNA product. While these techniques are less sensitive than sequencing, they are simpler to perform on a large number of samples. An example of a mismatch cleavage technique is the RNase protection method. In the practice of the present invention, the method involves the use of a labeled riboprobe that is complementary to a horse sequence comprising the common allele for a SNP. The riboprobe and either mRNA or DNA isolated from a physiological sample are annealed (hybridized) together and subsequently digested with the enzyme RNase A that is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA.

In similar fashion, DNA probes can be used to detect mismatches, through enzymatic or chemical cleavage. Alternatively, mismatches can be detected by shifts in the electrophoretic mobility of mismatched duplexes relative to matched duplexes. With either riboprobes or DNA probes, the cellular mRNA or DNA that might contain a variation can be amplified using PCR before hybridization.

Nucleic acid analysis via microchip technology is also applicable to the present invention.

DNA sequences comprising a given SNP that have been amplified by use of PCR may also be screened using allele-specific probes. These probes are nucleic acid oligomers, each of which contains the SNP. For example, one oligomer may be about 30 nucleotides in length. By use of such allele-specific probes, PCR amplification products can be screened to identify the presence of a particular allele at a given SNP. Hybridization of allele-specific probes with amplified sequences can be performed, for example, on a nylon filter. Hybridization to a particular probe under stringent hybridization conditions indicates the presence of the same variation in the tissue as in the allele-specific probe.

### Certain Definitions

As used herein, the phrase “physiological sample” is meant to refer to a biological sample obtained from a mammal that contains nucleic acid. For example, a physiological sample can be a sample collected from an individual horse, such as including, but not limited to, e.g., a cell sample, such as a blood cell, e.g., a lymphocyte, a peripheral blood cell; a tissue sample; an organ sample; a hair sample, e.g., a hair sample with roots; and/or a fluid sample, such as blood or semen.

An “allele” is a variant form of a particular gene/genetic location. For example, the present invention relates, inter alia, to the discovery that some alleles present at particular genetic locations are associated with a horse's ability to pace. The coexistence of multiple alleles at a locus is known as “genetic polymorphism.” Any site at which multiple alleles exist as stable components of the population is by definition “polymorphic.” An allele is defined as polymorphic if it is present at a frequency of at least 1% in the population. A “single nucleotide polymorphism (SNP)” is a DNA sequence variation that involves a change in a single nucleotide. Diploid organisms have two alleles at each genetic locus, with one allele inherited from each parent. Each pair of alleles represents the genotype at a particular genetic location. Genotypes are described as homozygous if there are two identical alleles at a particular locus and as heterozygous if the two alleles differ.

“Oligonucleotide probe” can refer to a nucleic acid segment, such as a primer, that is useful to amplify a particular sequence, wherein the probe is complementary to, and hybridizes specifically to, the particular sequence, or to a nucleic acid region that flanks the particular sequence (e.g., a region that flanks a SNP of interest).

As used herein, the term “nucleic acid” and “polynucleotide” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

The terms “protein,” “peptide” and “polypeptide” are used interchangeably herein.

The invention encompasses isolated or substantially purified nucleic acid compositions. In the context of the present invention, an “isolated” or “purified” DNA molecule is a DNA molecule that, by human intervention, exists apart from its native environment and is therefore not a product of nature. An isolated DNA molecule may exist in a purified form or may exist in a non-native environment. For example, an “isolated” or “purified” nucleic acid molecule, or portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences and proteins or partial-length proteins encoded thereby are also encompassed by the present invention.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Synthetic” polynucleotides are those prepared by chemical synthesis.

“Recombinant DNA molecule” is a combination of DNA sequences that are joined together using recombinant DNA technology and procedures used to join together DNA sequences as described, for example, in Sambrook and Russell (2001).

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

“Somatic mutations” are those that occur only in certain tissues, e.g., in liver tissue, and are not inherited in the germline. “Germline” mutations can be found in any of a body's tissues and are inherited.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

Software for performing BLAST analyses is publicly available through the National Center for Biotechnology Information (see the World Wide Web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When using BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by a BLAST program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, or at least 80%, 90%, or even at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%; at least 80%, 81%, 82%, 83%, 84%, 85%, 86%, 87%, 88%, or 89%; or at least 90%, 91%, 92%, 93%, or 94%; or even at least 95%, 96%, 97%, 98% or 99% sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The Tₘ is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl:

Tₘ 81.5° C.+16.6 (log M)+0.41(% GC)−0.61(% form)−500/L

where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired T, those of ordinary skill will understand that variations in the stringency of hybridization and/or wash solutions are inherently described. If the desired degree of mismatching results in a T of less than 45° C. (aqueous solution) or 32° C. (formamide solution), it is preferred to increase the SSC concentration so that a higher temperature can be used. Generally, highly stringent hybridization and wash conditions are selected to be about 5° C. lower than the thermal melting point (Tₘ) for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Thus, the genes and nucleotide sequences of the invention include both the naturally occurring sequences as well as mutant forms.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms.”

A “host cell” is a cell which has been transformed, or is capable of transformation, by an exogenous nucleic acid molecule. Thus, “transformed,” “transgenic,” and “recombinant” refer to a host cell or organism into which a heterologous nucleic acid molecule has been introduced. The nucleic acid molecule can be stably integrated into the genome generally known in the art. Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically includes sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will have the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The transcriptional cassette will include in the 5′-3′ direction of transcription, a transcriptional and translational initiation region, a DNA sequence of interest, and a transcriptional and translational termination region functional in plants. The termination region may be native with the transcriptional initiation region, may be native with the DNA sequence of interest, or may be derived from another source.

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified through, for example, the use of single-stranded mutagenesis. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Genome” refers to the complete genetic material of an organism.

“Coding sequence” refers to a DNA or RNA sequence that codes for a specific amino acid sequence and excludes the non-coding sequences. For example, a DNA “coding sequence” or a “sequence encoding” a particular polypeptide, is a DNA sequence which is transcribed and translated into a polypeptide in vitro or in vivo when placed under the control of appropriate regulatory elements. The boundaries of the coding sequence are determined by a start codon at the 5′-terminus and a translation stop codon at the 3′-terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. It may constitute an “uninterrupted coding sequence,” i.e., lacking an intron, such as in a cDNA or it may include one or more introns bounded by appropriate splice junctions. An “intron” is a sequence of RNA that is contained in the primary transcript but that is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

The term “regulatory sequence” is art-recognized and intended to include promoters, enhancers and other expression control elements (e.g., polyadenylation signals). Such regulatory sequences are known to those skilled in the art. It should be understood that the design of the expression vector may depend on such factors as the choice of the host cell to be transfected and/or the amount of fusion protein to be expressed.

The term DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

A control element, such as a promoter, “directs the transcription” of a coding sequence in a cell when RNA polymerase binds the promoter and transcribes the coding sequence into mRNA, which is then translated into the polypeptide encoded by the coding sequence.

A cell has been “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to eukaryotic cells, a stably transformed cell is one in which the exogenous DNA has become integrated into the chromosome so that it is inherited by daughter cells through chromosome replication. This stability is demonstrated by the ability of the eukaryotic cell to establish cell lines or clones having a population of daughter cells containing the exogenous DNA.

“Operably-linked” refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” or “translation stop codon” or “stop codon” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. The change of at least one nucleotide in a nucleic acid sequence can result in an interruption of the coding sequence of the gene, e.g., a premature stop codon.

The invention will now be illustrated by the following non-limiting Examples.

### Example 1. Identification and Validation of Genetic Variants Predictive of Gait in Standardbred Horses

Several horse breeds have been specifically selected for the ability to exhibit alternative patterns of locomotion, or gaits. A premature stop codon in the gene DMRT3 is permissive for “gaitedness” across breeds. However, this mutation is nearly fixed in both American Standardbred trotters and pacers, which perform a diagonal (trot) and lateral (pace) gait, respectively, during harness racing. This suggests that modifying alleles must influence the preferred gait at high speeds in these populations. Accordingly, experiments were performed to identify putative modifying alleles associated with gait in a large cohort of Standardbred pacers and trotters using a combination of GWAS and variant discovery via whole-genome sequencing. Specifically, genome-wide association analysis for the ability to pace was performed in 542 Standardbred horses (n=176 pacers, n=366 trotters) with genotype data imputed to ˜74,000 single nucleotide polymorphisms (SNPs). Nineteen SNPs on nine chromosomes (ECA1, 2, 6, 9, 17, 19, 23, 25, 31) reached genome-wide significance (p<1.44×10′). Variant discovery in regions surrounding these SNPs and additional regions of interest was carried out via whole-genome sequencing. A set of 303 variants from 22 chromosomes with putative modifying effects on gait was genotyped in 659 Standardbreds (n=231 pacers, n=428 trotters) using a high-throughput Sequenom assay. Random forest classification analysis resulted in an out-of-box error rate of 0.61%. A conditional inference tree algorithm containing seven SNPs predicted status as a pacer or trotter with 99.1% accuracy and subsequently performed with 99.4% accuracy in an independently sampled population of 166 Standardbreds (n=83 pacers, n=83 trotters). This highly accurate algorithm could be used by owners/trainers to identify Standardbred horses with the potential to race as pacers prior to initiating training and would enable fine-tuning of breeding programs with designed matings. The algorithm may also be investigated for use in other gaited breeds. Additionally, these SNPs may be further examined to determine whether they play a physiologically functional role in the tendency to pace, or simply tag true functional alleles.

### Results

GWAS Analysis

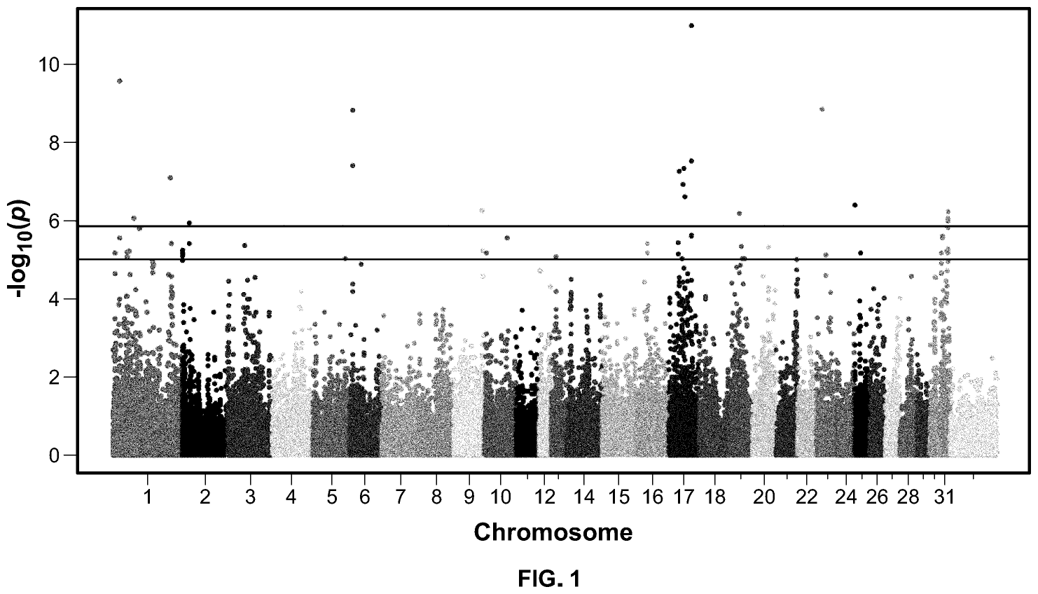

Horses included in the GWAS cohort (n=542) were genotyped on either the first generation (Equine SNP50; n=306) or second generation (Equine SNP70; n=236) Illumina equine beadchip. After genotype imputation, 73,691 markers were available for analysis; after pruning, 62,901 SNPs were included in the mixed model association analysis. After correction for relatedness and population structure, mixed model association analysis in GEMMA (Zhou X, Stephens M. Nat Genet. 2012; 44(7):821-4) revealed 19 SNPs on nine chromosomes that reached genome-wide significance (p<1.44×10-6, as determined by the likelihood ratio test) (FIG. 1, Table 1). Seven SNPs were located on equine (ECA) chromosome 17, three on ECA1, two on ECA6, two on ECA31, and one each on ECA2, 9, 19, 23, and 25. The ECA17 SNPs were from two distinct regions, 28.4-41.9 Mb (n=4) and 60.50-60.55 Mb (n=3) (Table 1). Only eight of the genome-wide significant SNPs were found within protein coding genes; all were intronic (Table 1). An additional 37 SNPs on 14 chromosomes reached significance considered to be suggestive for association with gait (p<1×10-5) (Consortium WTCC. Nature. 2007; 447(7145):661-78).

### Whole-Genome Sequencing

The results of whole-genome sequencing in this cohort was previously reported (McCoy et al., BMC Genomics. 2016; 17:41. doi: 10.1186/s12864-016-2385-z). Briefly, 12 individuals (6 pacers, 6 trotters) were sequenced at an average coverage depth of 6.4× (range 4.7×-7.9×). Six individuals (3 pacers, 3 trotters) were sequenced at an average coverage depth of 12.2× (range 10×-13.1×). After filtering, 14,588,812 variants were called, of which 13,157,608 were SNPs, 671,144 were insertions, and 760,060 were deletions. Of these variants, 99.1% were predicted to have no functional effect, 0.5% (85,916) were predicted to have minor functional effect, 0.4% (57,122) were predicted to have moderate functional effect, and 0.07% (9,662) were predicted to have major functional effect.

### Pooled Whole-Genome Sequencing

Two pools of genomic DNA (20 pacers, 20 trotters) were sequenced at a target depth of 30×. A total of eighty-nine 50 kb regions met one of the filtering criteria of either high differentiation between pacers and trotters (FST≥0.35) or a combination of low pool heterozygosity (Hp<0.1) in one of the groups and high differentiation (FST≥0.30). Some of these regions were contiguous or overlapping, creating larger regions of interest ranging in size from 75-300 kb. A total of 1,885 SNPs were called across all regions of interest. Of these, 1,273 were annotated by Ensembl (Equ Cab 2; GCA 000002305.1) as intergenic, 184 were located upstream of a gene, 138 were located downstream of a gene, 270 were located within an intron, and 20 were located within an exon.

### Sequenom Genotyping in the Discovery Cohort

Approximately 62,000 SNPs were evaluated from regions on 13 chromosomes identified as being of interest based on the GWA analysis (regions from 9 chromosomes that contained genome-wide significant SNPs, and regions from an additional 4 chromosomes that contained SNPs approaching genome-wide significance. An additional 1,885 SNPs were identified within regions of interest on 19 chromosomes from the pooled sequencing data. Three hundred three SNPs were included in the final Sequenom assay, including 190 SNPs from the whole-genome sequencing regions of interest (based on the GWA analysis) and 113 SNPs from the pooled sequencing regions of interest. Additionally, 98 ancestry informative markers (AIMs) were included on the assay to help control for population structure during downstream analysis (see, Methods and McCoy et al., BMC Genomics. 2016; 17:41. doi: 10.1186/s12864-016-2385-z).

Genotyping was performed in 720 individuals (n=458 trotters; n=262 pacers). After pruning, 245 SNPs were available for mixed model association analysis in GEMMA. With Bonferroni correction for multiple testing, statistical significance was set at p<2×10⁻⁴; after correcting for relatedness and population structure, 177 SNPs met this criteria for statistical significance (Table 2, Table 5). Pacers were more likely than trotters to carry the derived alleles (compared to the reference, a Thoroughbred) (Table 2). Nearly all of the trotters carried only a single copy of the alternate allele in each case, while it was more common for pacers to be homozygous for the alternate allele.

### Random Forest Classification Analysis

Random forest classification analysis of genotyping data from the Sequenom assay in 659 Standardbreds with racing records (n=428 trotters; n=231 pacers) yielded an out-of-box (00B) error rate of 0.61%, with a total of four misclassified individuals (three trotters misclassified as pacers, and one pacer misclassified as a trotter). Interestingly, one of the trotters who was predicted to be a pacer was in fact out of a line of pacing Standardbreds. There were 21 SNPs with a mean reduction of node impurity score (GINI index) >5 (Table 3). When the random forest analysis was repeated, the relative importance of these SNPs varied only slightly over multiple iterations. The most important SNPs for classification as a pacer or trotter according to this analysis were located on ECA1, 17, 23, and 30. Ten-fold cross-validation of this data using linear discriminate analysis resulted in a misclassification error of 0.0106.

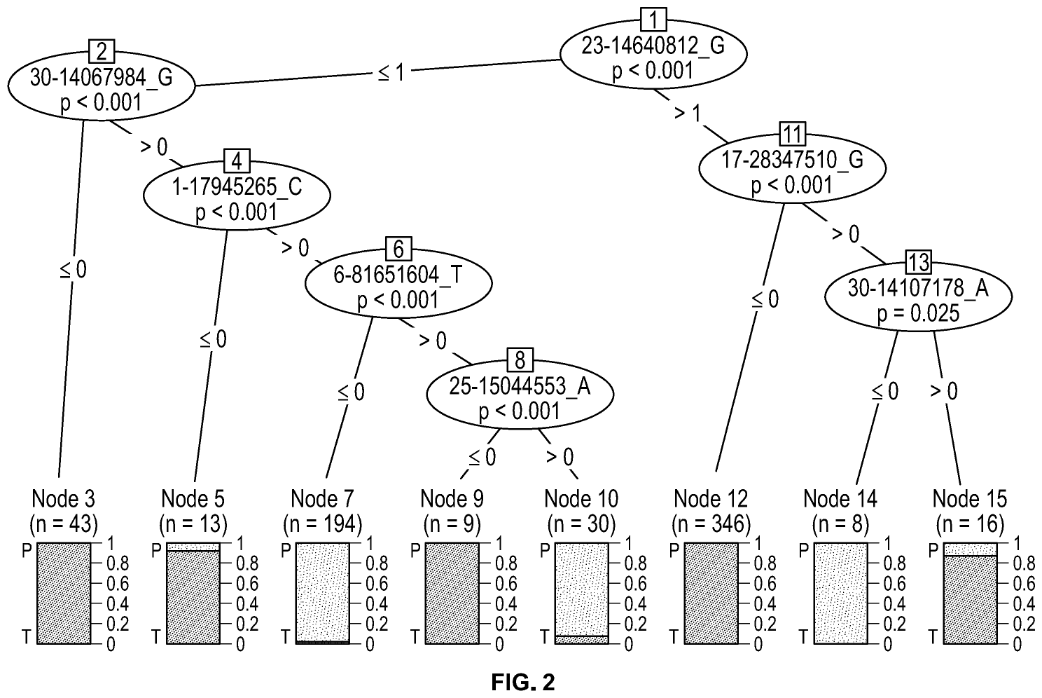

A conditional inference tree was constructed to determine the hierarchical organization of the most informative SNPs identified by random forest analysis. A tree composed of seven SNPs predicted status as a pacer or trotter among the 659 genotyped individuals with 99.1% accuracy, with only six horses misclassified (three pacers and three trotters). Again, one of these misclassified trotters came from a line of pacers. Considering pacing as the outcome of interest, this prediction model demonstrated a sensitivity of 98.7% (95% CI 96.25%-99.73%) and a specificity of 99.3% (95% CI 97.97%-99.86%). The seven SNPs were located on six chromosomes (ECA1, 6, 17, 23, 15, and 30) (FIG. 2). For four SNPs, the alternate allele was more common in pacers, and in three SNPs the alternate allele was more common in trotters. In either case, the group with the lower allele frequency included very few homozygotes (Table 4).

### Performance of the Prediction Algorithm in the Validation Cohort 188

One hundred sixty-six horses (n=83 trotters, n=83 pacers) were genotyped on the same custom Sequenom assay as the discovery cohort. The genotypes for the seven SNPs included in the conditional inference tree were extracted. Two individuals had missing genotypes at one or more of the SNPs and could not be classified. Of the remaining 164 horses, 163 were correctly classified as pacers or trotters, resulting in an overall accuracy of 99.4%. The single misclassified horse was a pacer.

### Discussion

The horse is unique among quadrupeds in that certain breeds have been strongly selected for the ability to exhibit alternative patterns of locomotion as a physiologic adaptation. Beyond giving insight into an economically important trait, improved understanding of the pathways that underlie alternative gaits in the horse may also provide insight into pathways that are dysregulated with disease in other species, as well as basic insight into the underlying neurobiology of locomotion. GWA analysis in the population identified 19 SNP markers that were associated with gait at a level of genome-wide significance. These SNPs defined regions of interest on nine chromosomes that contained more than two dozen named genes. However, a challenge arises in identifying biologically compelling candidate genes for gait because there is still much that is not known about the development of normal limb coordination. It is likely that many genes that play a role in the development of alternative gaits have not previously been associated with any aspect of neurobiology. This is aptly illustrated by DMRT3 which had initially been described as primarily playing a role in gonadal development and sexual differentiation (Kim et al., Gene Expr Patterns. 2003; 3(1):77-82). The DMRT3 nonsense mutation originally reported by Andersson et al. in 2012 (Nature. 2012; 488(7413):642-6) has now been reported to occur in 68 out of 141 breeds tested from around the world, and at high frequency (>50%) in all “gaited” breeds (Promerova et al., Anim Genet. 2014; 45(2):274-82). This example demonstrates that a strongly associated mutation cannot be ruled out as having a functional role in the development of alternative gaits simply because it falls within a gene that does not have a described role in neural development or locomotion.

As SNPs are chosen for inclusion in genotyping panels based on their distribution and frequency, rather than on predicted effect, it is unlikely that any of the markers in the GWA analysis play a functional role in gait. Rather, it is more likely that they are “tagging” truly functional variants with which they are in linkage disequilibrium (LD) (Spencer et al., PLoS Genet. 2009; 5(5):e1000477; Wall et al., Nat Rev Genet. 2003; 4(8):587-97). Standardbreds have been reported to have the greatest long-range LD (>1,200 kb) among horse breeds; thus, it is not unreasonable to expect that a significant SNP in a GWA might be “tagging” a functional sequence variant up to 1 Mb distant (or further). Given this, whole-genome sequencing was chosen as the most efficient way to catalogue variants within 1 Mb of the regions defined by the genome-wide significant SNPs. This approach also allowed variant discovery in a larger cohort of individuals (9 trotters and 9 pacers) than would have been feasible using a traditional candidate gene approach, giving a better picture of the alleles present in the population, as well as the segregation of these alleles with gait status. Pooled whole-genome sequencing offered a complementary approach to identifying regions and variants of interest based on population parameters of differentiation (FST) and heterozygosity (Hp). Pooled whole-genome sequencing has previously been used to identify genomic regions under selection, and identify candidate variants within those regions, in a number of plant and animal species (Carneiro et al., Science. 2014; 345(6200):1074-9; Woronik A, Wheat C W. J Evol Biol. 2017; 30(1):26-39; Fustier et al., Mol Ecol. 2017. doi: 10.1111/mec.14082).

Of the tens of thousands of SNPs discovered within regions of interest via whole-genome sequencing, only a small fraction could be selected for follow-up. Thus, it is still entirely possible that any truly functional alleles have not been identified despite the prioritization process. Indeed, of the top 40 variants from GEMMA analysis of Sequenom genotyping in 720 individuals, only two resulted in amino acid changes. However, given the strength of association of the selected variants with gait in this large population, it is highly likely that one or more of these genotyped variants are “tagging” specific functional alleles, and additional investigation of nearby variants is warranted. Future work to address the issue of functionality will include tissue expression profiles of the cerebellum and specific regions of the proximal cervical spinal cord that contain neuronal tracts known to play a role in coordinated locomotion.

Random forest classification analysis was selected to help prioritize among the numerous statistically significant variants in the Sequenom assay. In a random forest approach to a binary trait, the predicted probability of an individual expressing or not expressing a trait (in this case, being able to pace) is based on the aggregation of a number of decision trees (Bureau et al., Genet Epidemiol. 2005; 28(2):171-82; Pan et al., Genet Epidemiol. 2014; 38(3):209-19). Within these decision trees, each node is an attribute—in this case, the genotype at a given SNP. The importance of each SNP is determined by quantifying the increase of misclassified individuals when the genotype at that SNP is randomly permuted (Bureau et al., Genet Epidemiol. 2005; 28(2):171-82). This approach requires no prior knowledge of gene function and can accommodate multiple variants within the same gene. Random forest analysis has previously been successfully used to identify SNPs associated with feed intake in dairy cattle (Yao et al., J Dairy Sci. 2013; 96(10):6716-29), as well as pathway-phenotype associations in human bladder cancer (Pan et al., Genet Epidemiol. 2014; 38(3):209-19). In the instant population, random forest analysis revealed that just a few SNPs were of large importance in correctly classifying individuals, while a large number of SNPs were of minor to minimal importance. This suggested that an accurate prediction algorithm might be constructed, despite not knowing the functional importance of the individual SNPs. In fact, a prediction algorithm consisting of only seven SNPs was developed, which was >99% accurate in two independently sampled cohorts of Standardbreds. This is the first time that a prediction algorithm for gait has been reported and it could be used by owners/breeders/trainers for both marker-assisted selection and making training decisions by identifying young horses that have the genetic background to race successfully at the pace. This model may also be tested in other breeds to determine if its predictive value is specific to Standardbreds, to breeds that pace (e.g. Icelandic Horses), or if it is universally applicable across gaited breeds.

### Materials and Methods

### Study Population

This study was conducted under the approval of the University of Illinois (protocol 266 #15031) and University of Minnesota (protocol #1111B07193) Institutional Animal Care and Use Committees.

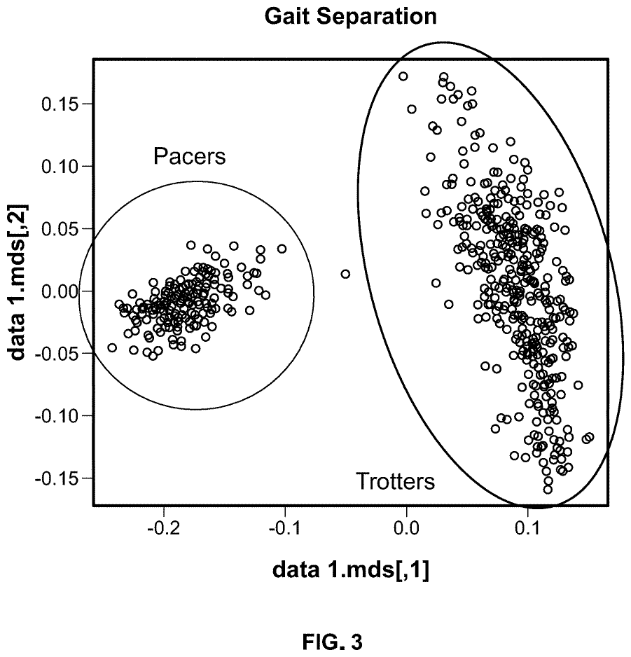

GWAS Cohort. The cohort for the genome-wide association study consisted of 542 Standardbred trotters (n=366) and pacers (n=176). All of the pacers and 153 trotters were from North America, while the remaining trotters were from Europe (Sweden, n=66; Norway, n=147). Horses were classified as pacers or trotters based on race records; if a horse never raced, their gait was assigned based on the race records of the sire and/or dam. Horses that raced as both pacers and trotters were classified as pacers. The North American and European trotters were related to each other. The pacers were genetically distinct from the trotters, with minimal admixture between groups (FIG. 3).

Sequenom Assay Discovery Cohort. Initially, 720 Standardbreds were genotyped on the custom Sequenom assay (see, Materials and Methods—Sequenom Assay), including the entire GWAS cohort. Horses were classified as pacers or trotters based on race records. Horses without a race record were excluded from downstream analysis, resulting in a final cohort comprised of 659 Standardbred trotters (n=428) and pacers (n=231).

Validation Cohort. The validation cohort was comprised of 166 independently sampled Standardbred trotters (n=83) and pacers (n=83) from North America. Horses were classified as pacers or trotters based on race records. These horses were genotyped on the custom Sequenom assay as described above, and the genotypes at the SNPs included in the prediction algorithm were extracted for analysis.

### DNA Isolation and Whole-Genome Genotyping

DNA was isolated from whole blood samples using the Gentra® Puregene® Blood Kit (Qiagen, Valencia, Calif.) per manufacturer recommendations. Briefly, RBC lysis solution was added to samples at a 3:1 ratio, incubated, and centrifuged. After discarding the supernatant, Cell lysis solution was added to the white blood cell pellet and the cells were re-suspended, after which protein was precipitated and discarded. DNA was precipitated in isopropanol and subsequently washed in ethanol prior to final hydration. Quantity and purity of extracted DNA were assessed using spectrophotometric readings at 260 and 280 nm (NanoDrop 1000, Thermo Scientific, Wilmington, Del.).

Genome-wide genotyping of single nucleotide polymorphism (SNP) markers was performed by Neogen GeneSeek (Lincoln, Nebr.) using an Illumina Custom Infinum SNP genotyping platform. Horses were genotyped at either 54,602 SNPs using the first generation Illumina Equine SNP50 chip (n=306), or at 65,157 SNPs using the second generation Illumina Equine SNP70 chip (n=236).

### Genotype Imputation

The two genotyping platforms used in the GWAS cohort share 45,703 SNPs. As an alternative to losing information from tens of thousands of SNPs by pruning to this shared marker list prior to merging files, genotype imputation may be used. This technique statistically estimates genotypes from non-assayed SNPs based on a comparison of haplotype blocks between one population and a second, more densely genotyped reference population. An established pipeline for imputation of equine genotyping data (McCoy A M and McCue M E. Anim Genet. 45(1):153 (2014)) was used to impute the 18,000 markers unique to the SNP70 chip in those horses genotyped on the SNP50 chip, and likewise to impute the 9,000 markers unique to the SNP50 chip in those horses genotyped on the SNP70 chip. Imputed files were merged for subsequent analysis using the—merge command in PLINK (Purcell et al., Am J Hum Genet. 2007; 81(3):559-75).

### Genome-Wide Association (GWA) Analysis

A GWA analysis with gait as the phenotype of interest was performed after genotype imputation using GEMMA (Genome-Wide Mixed Model Analysis) software (Zhou X, Stephens M. Nat Genet. 2012; 44(7):821-4). A centered relatedness matrix (−gk 2) was constructed using a LD-pruned set of markers (100 SNP windows, sliding by 25 SNPs along the genome, pruned at r²>0.2; PLINK command—indep-pairwise 100 25 0.2) (McCue et al., PLoS Genet. 2012; 8(1):e1002451). All three possible frequentists tests were performed: Wald, likelihood ratio, and score (−fa 4). A covariate file including sex and origin (North America or Europe) was incorporated into the mixed model (−c) and SNPs were pruned according to GEMMA default parameters (MAF<1%, missingness <95%). Association plots were generated using the base graphics package in the R statistical computing environment (R Core Team (2014). R: A language and environment for statistical computing. R Foundation for Statistical Computing, Vienna, Austria. URL http://www.R-project.org/). Genome-wide significance was set at p<1.44×10⁻⁶ based on the effective number of independent tests in the data (Li et al., Hum Genet. 2012; 131(5):747-56).

### Whole-Genome Sequencing

Whole-genome sequencing in this cohort has previously been reported (McCoy et al., BMC Genomics. 2016; 17:41. doi: 10.1186/s12864-016-2385-z, which is incorporated by reference herein in its entirety for all purposes). For the purposes of the previous study, horses were selected for sequencing based on haplotypes in regions of interest associated with osteochondrosis; however, they were also selected in a balanced manner for their gait phenotype, with 9 pacers and 9 trotters included. Briefly, genomic DNA (2-6 μg) from the 18 horses was submitted to the University of Minnesota Biomedical Genomics Center (UMGC) for quality control, library preparation, and sequencing. Samples were subjected to library preparation including fragmentation, polishing, and adaptor ligation, and were prepared with an indexed barcode for a 100 bp paired-end run on the Illumina HiSeq sequencer, per standard protocols. Targeted depth of coverage was 12× for six horses and 6× for twelve horses, with each group balanced for gait.

Data analysis, including quality control, alignment, and variant detection, was carried out following published best practices (Depristo et al., Nat Genet. 2011; 43(5):491-8; Van der Auwera et al., Curr Protoc Bioinformatics. 2013; 43:11.10.1-33) within the Galaxy framework hosted by the Minnesota Supercomputing Institute. Briefly, reads that passed quality control were mapped to the reference sequence (EquCab 2.0, September 2007 (Wade et al., 2009; 326(5954):865-7)) using BWA for Illumina (Li H, Durbin R. Bioinformatics. 2009; 25(14):1754-60). Ambiguously mapped reads, low quality reads, and PCR duplicates were removed, after which reads were realigned around indels. Base quality recalibration was performed to remove systematic bias. This process was completed for the reads from each of the eight lanes for every individual before merging the mapped and recalibrated “lane-level” BAM files into a single “sample-level” file. Removal of duplicates and realignment around indels was repeated on the merged file. The eighteen sample-level files were merged into three groups of six, evenly divided between pacers and trotters, for the purposes of variant calling using the UnifiedGenotyper utility of the Broad Institute's Genome Analysis ToolKit (GATK)(McKenna et al., Genome Res. 2010; 20(9):1297-303) with a threshold phred-scale score of 20.0. Variants were filtered using the following thresholds: Quality Depth (QD)<2.0 (assesses variant quality score taking into account depth of coverage at that variant), Read Position Rank Sum <−20.0 (Mann-Whitney Rank Sum test on the distance of the variant from the end of each read covering it), Fisher Strand (FS) >200.0 (phred-scaled p-value to detect strand bias). Filtered variant lists from the three groups were combined into a single variant calling file (VCF) for subsequent analysis. Predicted functional effect for each called variant was determined based on the current equine reference genome annotation using SnpEff (Cingolani et al., Fly (Austin). 2012; 6(2):80-92). Frequency of variants within cases and controls, and the significance of frequency differences, was calculated using SnpSift CaseControl (Cingolani et al., Front Genet. 2012; 3:35). Variants from particular chromosomal regions of interest were selected using SnpSift Intervals and converted into Excel format for further evaluation.

### Pooled Whole-Genome Sequencing

Genomic DNA from 20 pacers and 20 trotters were combined into two pools, each comprising equimolar amounts of DNA from all individuals, for the purpose of whole-genome sequencing. These individuals were selected from the GWAS cohort and were chosen to be as unrelated as possible on the basis of coancestry coefficient generated from the whole-genome genotyping data (PLINK command—genome). Selected pacers had coancestry coefficients <0.06 (no more closely related than first cousins); selected trotters had coancestry coefficients <0.14 (one pair of half-siblings, the rest less closely related).

The two DNA pools were sequenced to 30× average depth of coverage using an Illumina HiSeq2500 sequencer at Uppsala University. The resulting paired reads were subjected to sequencing adaptor trimming and were subsequently aligned to the horse reference genome (EquCab2.1)(Wade et al., 2009; 326(5954):865-7) using the Burrows-Wheeler alignment algorithm as implemented in BWA for Illumina (Li H, Durbin R. Bioinformatics. 2009; 25(14):1754-60) (bwa sampe) using default alignment settings. Aligned reads were subjected to duplicate removal using the algorithm MarkDuplicates implemented in the Picard-tools software (http://broadinstitute/github.io/picard). SNP and small insertion/deletion calling was performed using the UnifiedGenotyper algorithm of the Genome Analysis Toolkit (GATK) (McKenna et al., Genome Res. 2010; 20(9):1297-303), and the resulting SNP calls were filtered using published best practice variant filtration settings (Depristo et al., Nat Genet. 2011; 43(5):491-8; Van der Auwera et al., Curr Protoc Bioinformatics. 2013; 43:11.10.1-33). Numbers of sequence reads corresponding to the reference and variant alleles at filtered SNP sites were determined and were used to estimate allele frequencies for the pacer and trotter pools at each SNP locus. The allele counts and frequencies were then used to calculate the fixation index (FST) for the contrast between the two pools and to calculate estimated pool heterozygosity (Hp) within each pool for 50% overlapping sliding windows of 50 kilobases along the genome as previously described (Carneiro et al., Science. 2014; 345(6200):1074-9). Distributions of the genome-wide FST and Hp values were consulted to determine the genomic intervals displaying the most strongly differentiated loci between the pools and the most strongly fixed loci within each pool, respectively. From these distributions a strategy was used where windows fulfilling one of two criteria, (1) FST≥0.35 or (2) FST≥0.30 with the additional criteria of at least one pool showing Hp<0.1, were selected as regions of interest.

### Sequenom Assay

A custom Sequenom genotyping assay was designed for high-throughput evaluation of prioritized variants. Variants were selected from top regions of interest identified in the GWA, as well as from regions from the pooled sequencing data with high differentiation between pacers and trotters (FST≥0.35) or a combination of low pool heterozygosity (Hp<0.1) in one of the groups and high differentiation (FST>0.30). SNPs discovered via whole-genome sequencing that passed quality control filters were prioritized according to the following parameters: 1) segregation with gait (preferentially with the alternate allele found in all or nearly all pacers and less than half of the trotters); 2) not intergenic; 3) non-synonymous, then synonymous changes; 4) if intronic, close to the exon-intron border (preferably <100 bp); 5) coding genes preferred over non-coding; and 6) if upstream/downstream, as close as possible to start/stop codon. Variants from pooled sequencing data were prioritized according to criteria 2-6.

When possible, at least one variant was selected from each coding gene within each region of interest. Among adjacent variants with equal magnitude of predicted functional effect, the one with the higher genomic p-value was selected for inclusion. Ancestry informative markers (AIMs) were also included in the assay to help control for population structure (Petersen et al., Plant and Animal Genome Conference XX; 2012 Jan. 14-18, 2012; San Diego, Calif.). These 98 markers have previously been reported (McCoy et al., BMC Genomics. 2016; 17:41. doi: 10.1186/s12864-016-2385-z); they describe more than 97% of the genetic variation captured by principal components from genome-wide genotyping data in the Standardbred breed.

### Variant Analysis

Mixed Model Association Analysis. Genotyping data from the Sequenom assay were pruned using default parameters in GEMMA (MAF<1%, missingness <95%). The mixed model included a sex covariate (−c) and a relatedness matrix constructed from the AIMs (−gk 2). All three possible frequentist tests were calculated as described under Genome-Wide Association (GWA) Analysis. Statistical significance was set at p<0.05 with a Bonferroni correction for multiple testing.

Random Forest Classification Analysis. Genotyping data from the Sequenom assay from 659 horses with race records (see Sequenom Assay Discovery Cohort) were subjected to random forest classification analysis using the ‘randomForest’ function of the ‘randomForest’ package in R (Liaw A, Wiener M., R News. 2002; 2:18-22). The default parameters were used for number of trees (500) and number of variables tried at each split (√n, where n is the total number of variables). SNPs were pruned for missingness <90% prior to analysis. These data were subsequently subjected to 10-fold cross-validation using linear discriminate analysis to estimate misclassification error using the ‘errorest’ function of the ‘ipred’ package in R (Peters A, Hothorn T. ipred: Improved Predictors. R package version 0.9-5. 2015. Available from: http://CRAN.R-project.org/package=ipred). To determine the hierarchical organization of the most informative SNPs identified by random forest analysis, a conditional inference tree was constructed using the ‘ctree’ function of the ‘randomForest’ package.

Validation of Predictive Algorithm. As described under Validation Cohort, 166 independently sampled Standardbreds were genotyped on the custom Sequenom assay, and the genotypes at the SNPs included in the conditional inference tree were extracted for analysis. Horses were classified as pacers or trotters, observing the hierarchical relationships of the seven predictive SNPs as dictated by the conditional inference tree, by an individual blinded to their true gait status. Subsequently, the predicted gait was compared to the true gait, and sensitivity, specificity, and overall accuracy of the prediction algorithm was calculated.

### Tables

TABLE 1
Genome-wide significant (p < 1.44 × 10⁻⁶) SNPs from GEMMA mixed model
analysis in 542 Standardbred pacers and trotters (sex and origin covariates). After pruning,
analysis included 62,901 SNPs. Uncorrected p-values
are presented for the Likelihood ratio test (LRT).

| CHR | Region | BP | P_LRT | Location | Genes in Region |
|---|---|---|---|---|---|
| 1 | 18.1 Mb | 18091577 | 9.1E−10 | Intergenic | CASP7, NRAP |
| 1 | 55.3 Mb | 55259288 | 1.4E−06 | intron 3 | CTNNA3 |
| 1 | 155.2 Mb | 155226154 | 2.0E−07 | Intergenic | OR4Q3, OR11H1-like, OR11G2- |
| | | | | | like |
| 2 | 19.7 Mb | 19755735 | 1.4E−06 | intron 2 | SFA 3 |
| 6 | 7.0-7.4 Mb | 7000504 | 3.3E−08 | Intergenic | no named or predicted genes |
| | | 7372690 | 9.6E−10 | Intergenic | |
| 9 | 76.3 Mb | 76324169 | 8.2E−07 | Intergenic | no named or predicted genes |
| 17 | 28.4-41.9 Mb | 28460851 | 2.7E−07 | intron 26 | VWA8 |
| | | 36291973 | 2.8E−07 | Intergenic | NAA16, MTRF1 , KBTBD7, |
| | | 40999944 | 1.3E−07 | Intergenic | WBP4, ELF1, SUGT1, CNMD, |
| | | | | | PCDH8, OLFM4, PCGF5, |
| | | | | | RGCC, KBTBD6, PCDH17, |
| | | | | | RPL7A, DIAPH3, TDRD3, |
| | | | | | PCDH20, TDGF1, ATP6V1G3, |
| | | | | | PCDH9, |
| | | 41905502 | 2.0E−07 | Intergenic | ENSECAG00000001575, |
| | | | | | ENSECAG00000016249, |
| | | | | | ENSECAG00000016703, |
| | | | | | ENSECAG00000001672, |
| | | | | | ENSECAG00000001728, |
| | | | | | ENSECAG00000001792 |
| 17 | 60.50-60.55 Mb | 60503138 | 5.2E−08 | Intergenic | ENSECAG00000002865 |
| | | 60523882 | 5.2E−08 | Intergenic | |
| | | 60554458 | 3.9E−11 | Intergenic | |
| 19 | 24.64 Mb | 24644810 | 4.6E−07 | intron 5 | DNAJB11 |
| 23 | 14.6 Mb | 14650375 | 1.9E−09 | intron 1 | PRUNE2 |
| 25 | 2 Mb | 2021044 | 2.2E−07 | intron 8 | PAX5 |
| 31 | 18.19-18.20 Mb | 18194086 | 9.6E−07 | intron 2 | SASH1 |
| | | 18200337 | 7.1E−07 | intron 1 | |

CHR = chromosome; BP = base pair.
Gene annotations are from Ensembl (Equ Cab 2; GCA_000002305.1). Genes that were predicted, but unnamed, in Ensembl were identified via BLAST (https://blast.ncbi.nlm.nih.gov/Blast.cgi) when possible.

TABLE 2
Summary of top 40 SNPs from GEMMA mixed model analysis in 720
Standardbred pacers and trotters genotyped on the
custom Sequenom assay, including frequency
of the alternate allele in pacers and trotters at each
SNP. After pruning, analysis included 245
SNPs. Uncorrected p-values are presented
for the Likelihood ratio test (lrt). A summary of all statistically
significant SNPs from this analysis, including results from all three
frequentist tests performed, can be found in Table 5.

| | | | | Pacer | Trotter |
|---|---|---|---|---|---|
| Rank | CHR | BP | p_lrt | Freq | Freq |
| 1 | 30 | 14067984 | 1.4E−38 | 0.93 | 0.22 |
| 2 | 30 | 14107178 | 2.2E−38 | 0.08 | 0.79 |
| 3 | 17 | 28540291 | 2.3E−38 | 0.70 | 0.07 |
| 4 | 17 | 28361747 | 4.0E−37 | 0.58 | 0.02 |
| 5 | 23 | 14640812 | 1.0E−36 | 0.20 | 0.92 |
| 6 | 23 | 14648590 | 1.1E−36 | 0.80 | 0.09 |
| 7 | 23 | 14649864 | 1.2E−36 | 0.21 | 0.92 |
| 8 | 30 | 14947553 | 3.7E−33 | 0.07 | 0.74 |
| 9 | 1 | 35729338 | 9.7E−33 | 0.70 | 0.08 |
| 10 | 1 | 35731849 | 1.1E−32 | 0.70 | 0.08 |
| 11 | 1 | 35731283 | 1.3E−32 | 0.70 | 0.08 |
| 12 | 1 | 35726345 | 1.9E−32 | 0.71 | 0.09 |
| 13 | 1 | 35720250 | 1.9E−32 | 0.70 | 0.08 |
| 14 | 30 | 15055793 | 2.3E−32 | 0.93 | 0.25 |
| 15 | 30 | 15068782 | 2.5E−32 | 0.93 | 0.26 |
| 16 | 17 | 28347510 | 8.1E−31 | 0.56 | 0.02 |
| 17 | 30 | 14936139 | 1.1E−29 | 0.91 | 0.26 |
| 18 | 1 | 17945265 | 2.7E−29 | 0.93 | 0.24 |
| 19 | 1 | 35721326 | 5.3E−29 | 0.70 | 0.09 |
| 20 | 17 | 28458432 | 1.9E−28 | 0.71 | 0.15 |
| 21 | 30 | 15124747 | 1.4E−27 | 0.86 | 0.26 |
| 22 | 3 | 3051017 | 1.2E−26 | 0.76 | 0.15 |
| 23 | 17 | 28658966 | 2.5E−26 | 0.56 | 0.07 |
| 24 | 23 | 20652865 | 4.0E−26 | 0.82 | 0.14 |
| 25 | 23 | 20662320 | 6.6E−26 | 0.82 | 0.14 |
| 26 | 20 | 27691110 | 2.1E−24 | 0.63 | 0.07 |
| 27 | 1 | 48896092 | 6.6E−24 | 0.54 | 0.08 |
| 28 | 25 | 11811829 | 7.2E−22 | 0.77 | 0.16 |
| 29 | 1 | 38573734 | 9.2E−22 | 0.23 | 0.83 |
| 30 | 17 | 29271555 | 1.3E−21 | 0.55 | 0.05 |
| 31 | 25 | 11783623 | 1.6E−21 | 0.77 | 0.16 |
| 32 | 30 | 14059751 | 3.6E−21 | 0.15 | 0.69 |
| 33 | 17 | 28658850 | 4.5E−21 | 0.54 | 0.07 |
| 34 | 25 | 11800074 | 7.5E−21 | 0.76 | 0.16 |
| 35 | 1 | 38591441 | 2.6E−20 | 0.77 | 0.21 |
| 36 | 1 | 38592542 | 4.2E−19 | 0.77 | 0.22 |
| 37 | 23 | 14645077 | 1.8E−18 | 0.16 | 0.78 |
| 38 | 25 | 15839070 | 3.0E−18 | 0.83 | 0.21 |
| 39 | 1 | 38306816 | 7.0E−18 | 0.71 | 0.13 |
| 40 | 3 | 2494992 | 9.0E−18 | 0.70 | 0.18 |

CHR = chromosome; BP = base pair; hom = homozygous; het = heterozygous.

TABLE 3
Results of Random Forest analysis of 303 SNPs in 659 Standardbreds
with race scores are reported as a GINI index, reflecting reduction in
records. Importance node impurity. The higher the GINI
index, the more misclassification is introduced by random
permutation of the variable.

| CHR | BP | Allele | MeanDecreaseGini |
|---|---|---|---|
| 17 | 28347510 | G | 15.05209 |
| 30 | 14947553 | G | 13.06787 |
| 30 | 14067984 | G | 13.03264 |
| 1 | 17945265 | C | 12.19836 |
| 17 | 28361747 | G | 11.95995 |
| 30 | 14107178 | A | 11.37835 |
| 23 | 14648590 | A | 10.82176 |
| 23 | 14649864 | C | 10.18182 |
| 30 | 15068782 | G | 9.61353 |
| 23 | 14640812 | G | 9.09522 |
| 1 | 35731283 | T | 7.759352 |
| 23 | 20652865 | A | 7.559141 |
| 17 | 28540291 | A | 7.443625 |
| 30 | 15055793 | C | 6.630535 |
| 1 | 35731849 | G | 6.608307 |
| 1 | 35729338 | T | 6.585548 |
| 30 | 14936139 | A | 6.334448 |
| 23 | 20662320 | A | 5.789609 |
| 1 | 35720250 | C | 5.701313 |
| 1 | 35721326 | C | 5.494415 |
| 1 | 35726345 | T | 5.341378 |

CHR = chromosome; BP = base pair.

TABLE 4
Alternate allele frequencies for each of the 7 SNPs in the conditional
inference tree.

| | | | Pacer | Trotter |
|---|---|---|---|---|
| CHR | BP | Allele | Freq | Freq |
| 23 | 14640812 | G | 0.21 | 0.92 |
| 17 | 28347510 | G | 0.57 | 0.02 |
| 30 | 14107178 | A | 0.08 | 0.79 |
| 30 | 14067984 | G | 0.93 | 0.23 |
| 1 | 17945265 | C | 0.93 | 0.23 |
| 6 | 81651604 | T | 0.07 | 0.52 |
| 25 | 15044553 | A | 0.78 | 0.18 |

CHR = chromosome; BP = base pair; Horn = homozygote; Het = heterozygote.

TABLE 5
Summary of 177 statistically significant SNPs from GEMMA mixed model
analysis in 720 Standardbred pacers and trotters genotyped on the custom
Sequenom assay. After pruning, analysis included 245 SNPs.
Uncorrected p-values are presented for the Wald test, the
Likelihood ratio test (lrt) and the Score test.

| Rank | CHR | BP | p_wald | p_lrt | p_score |
|---|---|---|---|---|---|
| 1 | 30 | 14067984 | 4.9E−39 | 1.4E−38 | 3.1E−31 |
| 2 | 30 | 14107178 | 1.2E−38 | 2.2E−38 | 3.5E−31 |
| 3 | 17 | 28540291 | 1.3E−38 | 2.3E−38 | 3.6E−31 |
| 4 | 17 | 28361747 | 2.3E−37 | 4.0E−37 | 2.2E−30 |
| 5 | 23 | 14640812 | 1.9E−37 | 1.0E−36 | 7.5E−30 |
| 6 | 23 | 14648590 | 2.0E−37 | 1.1E−36 | 8.2E−30 |
| 7 | 23 | 14649864 | 2.6E−37 | 1.2E−36 | 7.9E−30 |
| 8 | 30 | 14947553 | 2.1E−33 | 3.7E−33 | 1.1E−27 |
| 9 | 1 | 35729338 | 4.8E−33 | 9.7E−33 | 2.4E−27 |
| 10 | 1 | 35731849 | 5.5E−33 | 1.1E−32 | 2.6E−27 |
| 11 | 1 | 35731283 | 6.7E−33 | 1.3E−32 | 3.0E−27 |
| 12 | 1 | 35726345 | 9.8E−33 | 1.9E−32 | 3.7E−27 |
| 13 | 1 | 35720250 | 9.5E−33 | 1.9E−32 | 3.9E−27 |
| 14 | 30 | 15055793 | 1.3E−32 | 2.3E−32 | 4.0E−27 |
| 15 | 30 | 15068782 | 1.4E−32 | 2.5E−32 | 4.3E−27 |
| 16 | 17 | 28347510 | 4.2E−31 | 8.1E−31 | 5.3E−26 |
| 17 | 30 | 14936139 | 6.0E−30 | 1.1E−29 | 3.1E−25 |
| 18 | 1 | 17945265 | 1.1E−29 | 2.7E−29 | 7.6E−25 |
| 19 | 1 | 35721326 | 2.8E−29 | 5.3E−29 | 1.1E−24 |
| 20 | 17 | 28458432 | 1.9E−28 | 1.9E−28 | 1.8E−24 |
| 21 | 30 | 15124747 | 1.2E−27 | 1.4E−27 | 8.9E−24 |
| 22 | 3 | 3051017 | 8.6E−27 | 1.2E−26 | 4.9E−23 |
| 23 | 17 | 28658966 | 2.4E−26 | 2.5E−26 | 6.8E−23 |
| 24 | 23 | 20652865 | 1.4E−26 | 4.0E−26 | 1.8E−22 |
| 25 | 23 | 20662320 | 2.1E−26 | 6.6E−26 | 2.7E−22 |
| 26 | 20 | 27691110 | 1.4E−24 | 2.1E−24 | 2.5E−21 |
| 27 | 1 | 48896092 | 6.7E−24 | 6.6E−24 | 4.4E−21 |
| 28 | 25 | 11811829 | 4.5E−22 | 7.2E−22 | 2.3E−19 |
| 29 | 1 | 38573734 | 5.6E−22 | 9.2E−22 | 2.9E−19 |
| 30 | 17 | 29271555 | 9.4E−22 | 1.3E−21 | 3.4E−19 |
| 31 | 25 | 11783623 | 1.1E−21 | 1.6E−21 | 4.3E−19 |
| 32 | 30 | 14059751 | 3.4E−21 | 3.6E−21 | 6.1E−19 |
| 33 | 17 | 28658850 | 4.0E−21 | 4.5E−21 | 7.7E−19 |
| 34 | 25 | 11800074 | 4.8E−21 | 7.5E−21 | 1.5E−18 |
| 35 | 1 | 38591441 | 1.9E−20 | 2.6E−20 | 3.6E−18 |
| 36 | 1 | 38592542 | 3.2E−19 | 4.2E−19 | 3.4E−17 |
| 37 | 23 | 14645077 | 1.1E−18 | 1.8E−18 | 1.3E−16 |
| 38 | 25 | 15839070 | 1.7E−18 | 3.0E−18 | 1.9E−16 |
| 39 | 1 | 38306816 | 5.1E−18 | 7.0E−18 | 3.4E−16 |
| 40 | 3 | 2494992 | 8.0E−18 | 9.0E−18 | 3.6E−16 |
| 41 | 1 | 38592096 | 7.7E−18 | 1.0E−17 | 4.6E−16 |
| 42 | 16 | 28786474 | 9.3E−18 | 1.1E−17 | 4.3E−16 |
| 43 | 1 | 49602985 | 1.0E−16 | 1.0E−16 | 2.5E−15 |
| 44 | 25 | 15044553 | 1.0E−16 | 1.5E−16 | 4.6E−15 |
| 45 | 25 | 13031714 | 2.9E−16 | 2.9E−16 | 5.7E−15 |
| 46 | 1 | 38563255 | 4.1E−16 | 5.2E−16 | 1.1E−14 |
| 47 | 16 | 59391893 | 1.2E−15 | 1.2E−15 | 2.0E−14 |
| 48 | 17 | 51017875 | 1.2E−15 | 1.4E−15 | 2.4E−14 |
| 49 | 25 | 15845420 | 1.1E−15 | 1.6E−15 | 3.3E−14 |
| 50 | 16 | 59382124 | 2.1E−15 | 2.2E−15 | 3.3E−14 |
| 51 | 16 | 59352686 | 2.9E−15 | 3.0E−15 | 4.2E−14 |
| 52 | 20 | 27711111 | 3.3E−15 | 3.6E−15 | 5.1E−14 |
| 53 | 17 | 27685585 | 4.5E−15 | 4.7E−15 | 6.3E−14 |
| 54 | 25 | 16820893 | 5.3E−15 | 6.2E−15 | 8.7E−14 |
| 55 | 25 | 11793191 | 6.6E−15 | 8.5E−15 | 1.2E−13 |
| 56 | 20 | 25113918 | 1.9E−14 | 2.2E−14 | 2.6E−13 |
| 57 | 25 | 11689674 | 1.8E−14 | 2.3E−14 | 2.8E−13 |
| 58 | 3 | 2521561 | 2.5E−14 | 2.7E−14 | 2.9E−13 |
| 59 | 20 | 27650699 | 3.3E−14 | 3.6E−14 | 3.6E−13 |
| 60 | 3 | 53721793 | 6.1E−14 | 6.7E−14 | 6.3E−13 |
| 61 | 11 | 57422057 | 6.4E−14 | 7.1E−14 | 6.7E−13 |
| 62 | 11 | 36714823 | 8.0E−14 | 8.4E−14 | 7.4E−13 |
| 63 | 25 | 14418173 | 7.5E−14 | 9.4E−14 | 9.3E−13 |
| 64 | 11 | 36608682 | 1.4E−13 | 1.5E−13 | 1.3E−12 |
| 65 | 1 | 1.07E+08 | 1.6E−13 | 1.7E−13 | 1.4E−12 |
| 66 | 4 | 9116614 | 1.6E−13 | 2.0E−13 | 1.8E−12 |
| 67 | 1 | 18065598 | 2.4E−13 | 2.5E−13 | 1.8E−12 |
| 68 | 25 | 11691308 | 1.9E−13 | 2.5E−13 | 2.3E−12 |
| 69 | 1 | 38646291 | 2.5E−13 | 2.7E−13 | 2.0E−12 |
| 70 | 23 | 14980071 | 3.7E−13 | 3.5E−13 | 2.3E−12 |
| 71 | 25 | 3694284 | 4.8E−13 | 4.7E−13 | 3.1E−12 |
| 72 | 25 | 3657454 | 6.6E−13 | 6.4E−13 | 4.0E−12 |
| 73 | 1 | 18109069 | 9.9E−13 | 9.9E−13 | 6.0E−12 |
| 74 | 25 | 3724550 | 1.2E−12 | 1.1E−12 | 6.7E−12 |
| 75 | 25 | 16817685 | 1.3E−12 | 1.4E−12 | 8.2E−12 |
| 76 | 15 | 10100242 | 1.5E−12 | 1.6E−12 | 9.6E−12 |
| 77 | 20 | 27727105 | 2.7E−12 | 2.7E−12 | 1.5E−11 |
| 78 | 20 | 25103556 | 2.3E−12 | 2.8E−12 | 1.7E−11 |
| 79 | 6 | 81651604 | 5.8E−12 | 6.3E−12 | 3.2E−11 |
| 80 | 6 | 81299480 | 9.0E−12 | 9.4E−12 | 4.5E−11 |
| 81 | 20 | 27768899 | 9.9E−12 | 1.1E−11 | 5.1E−11 |
| 82 | 3 | 2506253 | 1.1E−11 | 1.1E−11 | 4.7E−11 |
| 83 | 11 | 36669528 | 1.1E−11 | 1.2E−11 | 5.5E−11 |
| 84 | 23 | 20658789 | 1.2E−11 | 1.3E−11 | 5.7E−11 |
| 85 | 25 | 15829342 | 1.2E−11 | 1.5E−11 | 7.7E−11 |
| 86 | 9 | 75816548 | 1.9E−11 | 2.0E−11 | 8.3E−11 |
| 87 | 6 | 81668230 | 2.0E−11 | 2.1E−11 | 9.2E−11 |
| 88 | 14 | 1388861 | 2.2E−11 | 2.1E−11 | 8.6E−11 |
| 89 | 1 | 17548101 | 3.3E−11 | 3.4E−11 | 1.3E−10 |
| 90 | 25 | 3666056 | 4.5E−11 | 4.2E−11 | 1.6E−10 |
| 91 | 25 | 3860478 | 5.6E−11 | 5.5E−11 | 2.0E−10 |
| 92 | 23 | 14814635 | 6.8E−11 | 6.5E−11 | 2.3E−10 |
| 93 | 14 | 1427118 | 7.1E−11 | 6.8E−11 | 2.4E−10 |
| 94 | 3 | 2489598 | 8.1E−11 | 7.6E−11 | 2.6E−10 |
| 95 | 1 | 17552161 | 1.1E−10 | 1.2E−10 | 4.0E−10 |
| 96 | 2 | 19698739 | 7.1E−11 | 1.4E−10 | 7.6E−10 |
| 97 | 2 | 19714056 | 8.1E−11 | 1.6E−10 | 9.2E−10 |
| 98 | 16 | 25722825 | 1.7E−10 | 1.7E−10 | 5.7E−10 |
| 99 | 11 | 50634935 | 1.6E−10 | 1.8E−10 | 6.5E−10 |
| 100 | 3 | 58070704 | 1.9E−10 | 1.9E−10 | 6.1E−10 |
| 101 | 25 | 15026761 | 2.0E−10 | 2.3E−10 | 8.1E−10 |
| 102 | 1 | 50226817 | 2.7E−10 | 2.7E−10 | 8.3E−10 |
| 103 | 24 | 10296168 | 4.5E−10 | 5.1E−10 | 1.7E−09 |
| 104 | 20 | 47062579 | 6.6E−10 | 6.7E−10 | 1.9E−09 |
| 105 | 14 | 1570169 | 7.0E−10 | 6.9E−10 | 1.9E−09 |
| 106 | 3 | 58077312 | 1.1E−09 | 1.3E−09 | 3.8E−09 |
| 107 | 1 | 35670985 | 1.4E−09 | 1.6E−09 | 4.4E−09 |
| 108 | 25 | 14531198 | 1.6E−09 | 1.7E−09 | 4.6E−09 |
| 109 | 24 | 10299566 | 1.6E−09 | 1.8E−09 | 5.2E−09 |
| 110 | 14 | 1368081 | 2.1E−09 | 2.0E−09 | 5.0E−09 |
| 111 | 3 | 53834511 | 2.1E−09 | 2.0E−09 | 5.0E−09 |
| 112 | 20 | 47092658 | 2.8E−09 | 2.8E−09 | 7.0E−09 |
| 113 | 9 | 75803120 | 2.9E−09 | 3.0E−09 | 7.7E−09 |
| 114 | 11 | 36775489 | 3.1E−09 | 3.2E−09 | 7.9E−09 |
| 115 | 29 | 3291497 | 4.4E−09 | 4.9E−09 | 1.2E−08 |
| 116 | 19 | 31393832 | 5.8E−09 | 6.3E−09 | 1.6E−08 |
| 117 | 2 | 19724672 | 3.6E−09 | 6.5E−09 | 2.4E−08 |
| 118 | 19 | 21446218 | 7.4E−09 | 7.7E−09 | 1.8E−08 |
| 119 | 24 | 10285906 | 1.6E−08 | 1.7E−08 | 3.8E−08 |
| 120 | 1 | 39589062 | 2.3E−08 | 2.5E−08 | 5.4E−08 |
| 121 | 11 | 58376457 | 2.3E−08 | 2.5E−08 | 5.5E−08 |
| 122 | 23 | 14813929 | 7.7E−08 | 7.4E−08 | 1.3E−07 |
| 123 | 5 | 66187039 | 9.0E−08 | 1.1E−07 | 2.3E−07 |
| 124 | 17 | 61744016 | 1.7E−07 | 1.7E−07 | 3.1E−07 |
| 125 | 3 | 49601762 | 2.5E−07 | 2.5E−07 | 4.4E−07 |
| 126 | 3 | 49488838 | 4.1E−07 | 4.1E−07 | 6.8E−07 |
| 127 | 2 | 18987527 | 5.4E−07 | 5.2E−07 | 8.2E−07 |
| 128 | 3 | 49857369 | 6.3E−07 | 6.1E−07 | 9.6E−07 |
| 129 | 11 | 29599837 | 6.6E−07 | 6.7E−07 | 1.1E−06 |
| 130 | 3 | 49601886 | 6.6E−07 | 6.8E−07 | 1.1E−06 |
| 131 | 3 | 49530033 | 6.8E−07 | 7.2E−07 | 1.2E−06 |
| 132 | 25 | 12791659 | 7.6E−07 | 7.4E−07 | 1.2E−06 |
| 133 | 2 | 18364832 | 8.3E−07 | 8.9E−07 | 1.5E−06 |
| 134 | 17 | 61728019 | 8.9E−07 | 9.3E−07 | 1.5E−06 |
| 135 | 11 | 29564206 | 9.4E−07 | 9.6E−07 | 1.5E−06 |
| 136 | 25 | 14737344 | 1.1E−06 | 1.1E−06 | 1.7E−06 |
| 137 | 3 | 49857478 | 1.1E−06 | 1.1E−06 | 1.8E−06 |
| 138 | 2 | 19775173 | 1.2E−06 | 1.1E−06 | 1.7E−06 |
| 139 | 17 | 61749334 | 1.3E−06 | 1.3E−06 | 2.0E−06 |
| 140 | 11 | 29532466 | 1.6E−06 | 1.6E−06 | 2.5E−06 |
| 141 | 25 | 12758770 | 1.7E−06 | 1.7E−06 | 2.5E−06 |
| 142 | 17 | 61717590 | 2.0E−06 | 2.1E−06 | 3.2E−06 |
| 143 | 3 | 56755586 | 2.1E−06 | 2.1E−06 | 3.1E−06 |
| 144 | 3 | 52318025 | 2.0E−06 | 2.2E−06 | 3.5E−06 |
| 145 | 3 | 57929520 | 2.3E−06 | 2.3E−06 | 3.3E−06 |
| 146 | 23 | 14182456 | 2.6E−06 | 2.5E−06 | 3.6E−06 |
| 147 | 3 | 58434545 | 2.5E−06 | 2.5E−06 | 3.7E−06 |
| 148 | 25 | 14760167 | 3.6E−06 | 3.7E−06 | 5.4E−06 |
| 149 | 25 | 14735220 | 5.2E−06 | 5.3E−06 | 7.6E−06 |
| 150 | 3 | 52680823 | 6.7E−06 | 6.7E−06 | 9.4E−06 |
| 151 | 12 | 16262259 | 7.9E−06 | 7.7E−06 | 1.0E−05 |
| 152 | 1 | 43245806 | 9.6E−06 | 9.3E−06 | 1.2E−05 |
| 153 | 25 | 13050176 | 1.1E−05 | 1.1E−05 | 1.5E−05 |
| 154 | 25 | 13047191 | 1.2E−05 | 1.2E−05 | 1.6E−05 |
| 155 | 2 | 18538576 | 1.3E−05 | 1.2E−05 | 1.6E−05 |
| 156 | 25 | 13021802 | 1.2E−05 | 1.2E−05 | 1.6E−05 |
| 157 | 25 | 13052616 | 1.4E−05 | 1.3E−05 | 1.8E−05 |
| 158 | 17 | 28485796 | 1.4E−05 | 1.3E−05 | 1.8E−05 |
| 159 | 3 | 56561263 | 1.4E−05 | 1.4E−05 | 1.9E−05 |
| 160 | 1 | 50226814 | 1.7E−05 | 1.6E−05 | 2.1E−05 |
| 161 | 25 | 13031250 | 1.6E−05 | 1.6E−05 | 2.1E−05 |
| 162 | 12 | 16270318 | 1.7E−05 | 1.7E−05 | 2.2E−05 |
| 163 | 26 | 3315939 | 1.8E−05 | 1.7E−05 | 2.2E−05 |
| 164 | 24 | 10276151 | 1.7E−05 | 1.8E−05 | 2.5E−05 |
| 165 | 5 | 66199885 | 2.5E−05 | 2.7E−05 | 3.8E−05 |
| 166 | 1 | 5322242 | 3.7E−05 | 3.7E−05 | 4.6E−05 |
| 167 | 3 | 57629621 | 3.9E−05 | 3.9E−05 | 4.8E−05 |
| 168 | 26 | 3315959 | 4.1E−05 | 4.1E−05 | 5.1E−05 |
| 169 | 26 | 3315992 | 6.7E−05 | 6.6E−05 | 8.0E−05 |
| 170 | 25 | 13016645 | 7.3E−05 | 7.2E−05 | 8.8E−05 |
| 171 | 26 | 3315794 | 7.7E−05 | 7.6E−05 | 9.2E−05 |
| 172 | 11 | 31319004 | 1.0E−04 | 1.0E−04 | 1.2E−04 |
| 173 | 11 | 31303355 | 1.2E−04 | 1.2E−04 | 1.5E−04 |
| 174 | 9 | 75896366 | 1.3E−04 | 1.4E−04 | 1.7E−04 |
| 175 | 25 | 15621763 | 1.6E−04 | 1.5E−04 | 1.8E−04 |
| 176 | 6 | 6609510 | 1.9E−04 | 1.8E−04 | 2.1E−04 |
| 177 | 3 | 49785110 | 1.9E−04 | 1.9E−04 | 2.2E−04 |

CHR = chromosome; BP = base pair.

### Example 2. Summary of Predictive Value of SNP Combinations

The seven SNPs identified in Example 1, Table 4 were further evaluated to ascertain the predictive value of various combinations of these SNPs. Specifically, ten-fold cross-validation was performed using linear discriminate analysis to determine prediction accuracy of all possible combinations of the seven markers (single nucleotide polymorphisms; SNPs) that make up the complete prediction algorithm (see, Tables 6 and 7). There were 659 horses in the population being tested. The same seed was used for each permutation, allowing direct comparisons.

TABLE 6
Combinations of the Seven SNP markers

| | | | | | | | | No. | Misclassification |
|---|---|---|---|---|---|---|---|---|---|
| Permutation | SNP | SNP | SNP | SNP | SNP | SNP | SNP | SNPs | Error |
| 1 | Chr1 | Chr6 | Chr17 | Chr23 | Chr25 | Chr30a | Chr30b | 7 | 0.011 |
| 3 | Chr1 | | Chr17 | Chr23 | Chr25 | Chr30a | Chr30b | 6 | 0.011 |
| 6 | Chr1 | Chr6 | Chr17 | Chr23 | | Chr30a | Chr30b | 6 | 0.011 |
| 7 | Chr1 | Chr6 | Chr17 | Chr23 | Chr25 | | Chr30b | 6 | 0.011 |
| 8 | Chr1 | Chr6 | Chr17 | Chr23 | Chr25 | Chr30a | | 6 | 0.011 |
| 2 | | Chr6 | Chr17 | Chr23 | Chr25 | Chr30a | Chr30b | 6 | 0.012 |
| 4 | Chr1 | Chr6 | | Chr23 | Chr25 | Chr30a | Chr30b | 6 | 0.015 |
| 5 | Chr1 | Chr6 | Chr17 | | Chr25 | Chr30a | Chr30b | 6 | 0.026 |
| 14 | | Chr6 | Chr17 | Chr23 | Chr25 | Chr30a | | 5 | 0.011 |
| 27 | Chr1 | Chr6 | Chr17 | Chr23 | | | Chr30b | 5 | 0.011 |
| 28 | Chr1 | Chr6 | Chr17 | Chr23 | | Chr30a | | 5 | 0.011 |
| 9 | | | Chr17 | Chr23 | Chr25 | Chr30a | Chr30b | 5 | 0.012 |
| 13 | | Chr6 | Chr17 | Chr23 | Chr25 | | Chr30b | 5 | 0.012 |
| 17 | Chr1 | | Chr17 | Chr23 | | Chr30a | Chr30b | 5 | 0.012 |
| 18 | Chr1 | | Chr17 | Chr23 | Chr25 | | Chr30b | 5 | 0.012 |
| 19 | Chr1 | | Chr17 | Chr23 | Chr25 | Chr30a | | 5 | 0.012 |
| 21 | Chr1 | Chr6 | | Chr23 | | Chr30a | Chr30b | 5 | 0.012 |
| 29 | Chr1 | Chr6 | Chr17 | Chr23 | Chr25 | | | 5 | 0.012 |
| 23 | Chr1 | Chr6 | | Chr23 | Chr25 | Chr30a | | 5 | 0.015 |
| 12 | | Chr6 | Chr17 | Chr23 | | Chr30a | Chr30b | 5 | 0.017 |
| 15 | Chr1 | | | Chr23 | Chr25 | Chr30a | Chr30b | 5 | 0.017 |
| 22 | Chr1 | Chr6 | | Chr23 | Chr25 | | Chr30b | 5 | 0.017 |
| 10 | | Chr6 | | Chr23 | Chr25 | Chr30a | Chr30b | 5 | 0.021 |
| 26 | Chr1 | Chr6 | Chr17 | | Chr25 | Chr30a | | 5 | 0.021 |
| 25 | Chr1 | Chr6 | Chr17 | | Chr25 | | Chr30b | 5 | 0.026 |
| 16 | Chr1 | | Chr17 | | Chr25 | Chr30a | Chr30b | 5 | 0.029 |
| 11 | | Chr6 | Chr17 | | Chr25 | Chr30a | Chr30b | 5 | 0.03 |
| 24 | Chr1 | Chr6 | Chr17 | | | Chr30a | Chr30b | 5 | 0.03 |
| 20 | Chr1 | Chr6 | | | Chr25 | Chr30a | Chr30b | 5 | 0.035 |
| 34 | | | Chr17 | Chr23 | Chr25 | Chr30a | | 4 | 0.012 |
| 58 | Chr1 | Chr6 | | Chr23 | | | Chr30b | 4 | 0.012 |
| 33 | | | Chr17 | Chr23 | Chr25 | | Chr30b | 4 | 0.014 |
| 53 | Chr1 | | Chr17 | Chr23 | | Chr30a | | 4 | 0.014 |
| 32 | | | Chr17 | Chr23 | | Chr30a | Chr30b | 4 | 0.015 |
| 42 | | Chr6 | Chr17 | Chr23 | | | Chr30b | 4 | 0.015 |
| 43 | | Chr6 | Chr17 | Chr23 | | Chr30a | | 4 | 0.015 |
| 46 | Chr1 | | | Chr23 | | Chr30a | Chr30b | 4 | 0.015 |
| 52 | Chr1 | | Chr17 | Chr23 | | | Chr30b | 4 | 0.015 |
| 59 | Chr1 | Chr6 | | Chr23 | | Chr30a | | 4 | 0.015 |
| 48 | Chr1 | | | Chr23 | Chr25 | Chr30a | | 4 | 0.017 |
| 47 | Chr1 | | | Chr23 | Chr25 | | Chr30b | 4 | 0.018 |
| 54 | Chr1 | | Chr17 | Chr23 | Chr25 | | | 4 | 0.018 |
| 64 | Chr1 | Chr6 | Chr17 | Chr23 | | | | 4 | 0.018 |
| 38 | | Chr6 | | Chr23 | Chr25 | Chr30a | | 4 | 0.021 |
| 37 | | Chr6 | | Chr23 | Chr25 | | Chr30b | 4 | 0.023 |
| 44 | | Chr6 | Chr17 | Chr23 | Chr25 | | | 4 | 0.023 |
| 60 | Chr1 | Chr6 | | Chr23 | Chr25 | | | 4 | 0.023 |
| 62 | Chr1 | Chr6 | Chr17 | | | Chr30a | | 4 | 0.024 |
| 30 | | | | Chr23 | Chr25 | Chr30a | Chr30b | 4 | 0.026 |
| 61 | Chr1 | Chr6 | Chr17 | | | | Chr30b | 4 | 0.027 |
| 50 | Chr1 | | Chr17 | | Chr25 | | Chr30b | 4 | 0.029 |
| 51 | Chr1 | | Chr17 | | Chr25 | Chr30a | | 4 | 0.029 |
| 41 | | Chr6 | Chr17 | | Chr25 | Chr30a | | 4 | 0.03 |
| 49 | Chr1 | | Chr17 | | | Chr30a | Chr30b | 4 | 0.03 |
| 36 | | Chr6 | | Chr23 | | Chr30a | Chr30b | 4 | 0.033 |
| 40 | | Chr6 | Chr17 | | Chr25 | | Chr30b | 4 | 0.033 |
| 57 | Chr1 | Chr6 | | | Chr25 | Chr30a | | 4 | 0.033 |
| 55 | Chr1 | Chr6 | | | | Chr30a | Chr30b | 4 | 0.036 |
| 63 | Chr1 | Chr6 | Chr17 | | Chr25 | | | 4 | 0.036 |
| 31 | | | Chr17 | | Chr25 | Chr30a | Chr30b | 4 | 0.038 |
| 56 | Chr1 | Chr6 | | | Chr25 | | Chr30b | 4 | 0.038 |
| 39 | | Chr6 | Chr17 | | | Chr30a | Chr30b | 4 | 0.04 |
| 45 | Chr1 | | | | Chr25 | Chr30a | Chr30b | 4 | 0.046 |
| 35 | | Chr6 | | | Chr25 | Chr30a | Chr30b | 4 | 0.061 |
| 75 | Chr1 | | | Chr23 | | Chr30a | | 3 | 0.015 |
| 91 | | | Chr17 | Chr23 | | Chr30a | | 3 | 0.015 |
| 92 | | | Chr17 | Chr23 | | | Chr30b | 3 | 0.015 |
| 76 | Chr1 | | | Chr23 | | | Chr30b | 3 | 0.017 |
| 70 | Chr1 | | Chr17 | Chr23 | | | | 3 | 0.024 |
| 96 | | | | Chr23 | Chr25 | Chr30a | | 3 | 0.026 |
| 97 | | | | Chr23 | Chr25 | | Chr30b | 3 | 0.029 |
| 72 | Chr1 | | Chr17 | | | Chr30a | | 3 | 0.032 |
| 73 | Chr1 | | Chr17 | | | | Chr30b | 3 | 0.032 |
| 74 | Chr1 | | | Chr23 | Chr25 | | | 3 | 0.032 |
| 66 | Chr1 | Chr6 | | Chr23 | | | | 3 | 0.033 |
| 85 | | Chr6 | | Chr23 | | Chr30a | | 3 | 0.033 |
| 93 | | | Chr17 | | Chr25 | Chr30a | | 3 | 0.033 |
| 90 | | | Chr17 | Chr23 | Chr25 | | | 3 | 0.035 |
| 86 | | Chr6 | | Chr23 | | | Chr30b | 3 | 0.036 |
| 82 | | Chr6 | Chr17 | | | Chr30a | | 3 | 0.038 |
| 65 | Chr1 | Chr6 | Chr17 | | | | | 3 | 0.04 |
| 69 | Chr1 | Chr6 | | | | | Chr30b | 3 | 0.04 |
| 84 | | Chr6 | | Chr23 | Chr25 | | | 3 | 0.04 |
| 68 | Chr1 | Chr6 | | | | Chr30a | | 3 | 0.041 |
| 80 | | Chr6 | Chr17 | Chr23 | | | | 3 | 0.041 |
| 71 | Chr1 | | Chr17 | | Chr25 | | | 3 | 0.043 |
| 79 | Chr1 | | | | | Chr30a | Chr30b | 3 | 0.043 |
| 83 | | Chr6 | Chr17 | | | | Chr30b | 3 | 0.044 |
| 94 | | | Chr17 | | Chr25 | | Chr30b | 3 | 0.044 |
| 98 | | | | Chr23 | | Chr30a | Chr30b | 3 | 0.044 |
| 77 | Chr1 | | | | Chr25 | Chr30a | | 3 | 0.046 |
| 78 | Chr1 | | | | Chr25 | | Chr30b | 3 | 0.047 |
| 95 | | | Chr17 | | | Chr30a | Chr30b | 3 | 0.047 |
| 87 | | Chr6 | | | Chr25 | Chr30a | | 3 | 0.056 |
| 88 | | Chr6 | | | Chr25 | | Chr30b | 3 | 0.058 |
| 99 | | | | | Chr25 | Chr30a | Chr30b | 3 | 0.058 |
| 81 | | Chr6 | Chr17 | | Chr25 | | | 3 | 0.065 |
| 67 | Chr1 | Chr6 | | | Chr25 | | | 3 | 0.067 |
| 89 | | Chr6 | | | | Chr30a | Chr30b | 3 | 0.085 |
| 111 | | | Chr17 | Chr23 | | | | 2 | 0.041 |
| 102 | Chr1 | | | Chr23 | | | | 2 | 0.043 |
| 104 | Chr1 | | | | | Chr30a | | 2 | 0.043 |
| 116 | | | | Chr23 | | Chr30a | | 2 | 0.044 |
| 105 | Chr1 | | | | | | Chr30b | 2 | 0.046 |
| 113 | | | Chr17 | | | Chr30a | | 2 | 0.047 |
| 114 | | | Chr17 | | | | Chr30b | 2 | 0.049 |
| 117 | | | | Chr23 | | | Chr30b | 2 | 0.05 |
| 115 | | | | Chr23 | Chr25 | | | 2 | 0.052 |
| 118 | | | | | Chr25 | Chr30a | | 2 | 0.056 |
| 107 | | Chr6 | | Chr23 | | | | 2 | 0.059 |
| 119 | | | | | Chr25 | | Chr30b | 2 | 0.067 |
| 120 | | | | | | Chr30a | Chr30b | 2 | 0.079 |
| 101 | Chr1 | | Chr17 | | | | | 2 | 0.082 |
| 106 | | Chr6 | Chr17 | | | | | 2 | 0.084 |
| 110 | | Chr6 | | | | | Chr30b | 2 | 0.084 |
| 109 | | Chr6 | | | | Chr30a | | 2 | 0.088 |
| 100 | Chr1 | Chr6 | | | | | | 2 | 0.09 |
| 112 | | | Chr17 | | Chr25 | | | 2 | 0.091 |
| 103 | Chr1 | | | | Chr25 | | | 2 | 0.094 |
| 108 | | Chr6 | | | Chr25 | | | 2 | 0.103 |
| 126 | | | | | | Chr30a | | 1 | 0.079 |
| 123 | | | Chr17 | | | | | 1 | 0.087 |
| 121 | Chr1 | | | | | | | 1 | 0.091 |
| 127 | | | | | | | Chr30b | 1 | 0.096 |
| 124 | | | | Chr23 | | | | 1 | 0.118 |
| 125 | | | | | Chr25 | | | 1 | 0.159 |
| 122 | | Chr6 | | | | | | 1 | 0.187 |

The SNP at Chromosome 1 is 17945265_C. The SNP at Chromosome 6 is 81651604_T. The SNP at Chromosome 17 is 28347510_G. The SNP at Chromosome 23 is 14640812_G. The SNP at Chromosome 30, designated Chr30a, is 14067984_G. The SNP at Chromosome 30, designated Chr30b, is 14107178_A.

TABLE 7
Summary of SNP combinations

| Number of | Range of prediction | |
|---|---|---|
| SNPs in model | accuracy | Certain SNP combination |
| 7 | 99.9% | full model |
| 6 | 97.4-99.9% | A. 1, 17, 23, 25, 30a, 30b |
| | | B. 1, 6, 17, 23, 30a, 30b |
| | | C. 1, 6, 17, 23, 25, 30b |
| | | D. 1, 6, 17, 23, 25, 30a |
| 5 | 96.5-99.9% | A. 6, 17, 23, 25, 30a |
| | | B. 1, 6, 17, 23, 30b |
| | | C. 1, 6, 17, 23, 30a |
| 4 | 93.9-99.8% | A. 17, 23, 25, 30a |
| | | B. 1, 6, 23, 30b |
| 3 | 93.3-99.5% | A. 1, 23, 30a |
| | | B. 17, 23, 30a |
| | | C. 17, 23, 30b |
| 2 | 89.7-95.9% | A. 17, 23 |
| 1 | 81.3-92.1% | A. 30a |

30a = 30-14067984
30b = 30-14107178

Results for the single SNP predictive value were also validated by hand in a second, independent population of 166 horses. Prediction accuracy in this second population ranged from 81.9-92.7%, with the most accurate SNP being 30b. There was no pattern in the misclassified individuals (i.e. the same individuals were not misclassified when different SNP combinations were used for prediction).

The high predictive value of multiple combinations of SNPs suggests that these are “tagging” regions/genes that are part of an as-yet unidentified network controlling gait/pattern development in the horse.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for identifying a horse having the ability to pace, comprising:
1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1; and
2) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

2. The method of claim 1, comprising:
1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25; and
2) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

3. The method of any one of claims 1-2, further comprising obtaining a physiological sample from the horse, wherein the sample comprises nucleic acid.

4. A method, comprising:
1) obtaining or having obtained a physiological sample from a horse, wherein the physiological sample comprises nucleic acid;
2) assaying or having assayed the sample to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1; and
3) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

5. The method of claim 4, comprising:
2) assaying or having assayed the sample to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25; and
3) identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

6. The method of any one of claims 1-5, further comprising selecting and training the identified horse for racing.

7. The method of any one of claims 1-5, further comprising selecting and breeding the identified horse.

8. The method of any one of claims 1-5, further comprising obtaining a sperm or egg sample from the identified horse, wherein the sperm or egg sample is used in a breeding program.

9. A method for selecting and training a horse for racing, comprising:
1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1;
2) identifying or having identified the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected; and
3) training the identified horse for racing.

10. A method for selecting a horse for a breeding program, comprising:
1) assaying or having assayed a nucleic acid sample from the horse to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1;
2) identifying or having identified the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected; and
3) breeding the identified horse or obtaining sperm or egg sample from the identified horse.

11. The method of any one of claims 1-10, wherein the genotypes of a panel of SNPs are detected, and wherein the panel comprises two or more SNPs.

12. The method of claim 11, wherein the panel comprises 3 or more SNPs.

13. The method of claim 11, wherein the panel comprises 5 or more SNPs.

14. The method of claim 11, wherein the panel comprises 7 SNPs.

15. The method of claim 11, comprising assaying a combination of SNPs, wherein the combination is selected from a combination described in Table 6 or Table 7.

16. The method any one of claims 1-15, wherein the identifying step further comprises using a learning statistical classifier system to analyze the SNP genotypes.

17. The method of claim 16, wherein the learning statistical classifier system is a Random Forest system.

18. The method of claim 16 or 17, wherein the panel has a misclassification error of less than 10%.

19. The method of claim 18, wherein the panel has a misclassification error of less than 1%.

20. A method of detecting at least one genetic variation in a horse, comprising:
1) obtaining or having obtained a physiological sample from the horse, wherein the physiological sample comprises nucleic acid; and
2) assaying the sample to detect the genotype of at least one single nucleotide polymorphism (SNP) located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and/or at position 35726345 on ECA1.

21. The method of claim 20, comprising:
2) assaying sample to detect the genotype of at least one single nucleotide polymorphism (SNP) at position 14640812 on equine (ECA) chromosome 23, at position 28347510 on ECA17, at position 14107178 on ECA30, at position 14067984 on ECA30, at position 17945265 on ECA1, at position 81651604 on ECA6 and/or at position 15044553 on ECA25.

22. The method of claim 20 or 21, wherein the genotypes of a panel of SNPs are detected, and wherein the panel comprises two or more SNPs.

23. The method of claim 22, wherein the panel comprises a combination of SNPs selected from a combination described in Table 6 or Table 7.

24. A method of measuring a panel of single nucleotide polymorphisms (SNPs) in a horse to predict whether the horse has the ability to pace, comprising:
1) obtaining or having obtained a physiological sample from the horse, wherein the physiological sample comprises nucleic acid;
2) assaying the sample to detect the genotype of each SNP in the panel, wherein the panel comprises two or more SNPs selected from the group consisting of a SNP located at position 14640812 on equine (ECA) chromosome 23, at position 28347510 on ECA17, at position 14107178 on ECA30, at position 14067984 on ECA30, at position 17945265 on ECA1, at position 81651604 on ECA6, at position 15044553 on ECA25, at position 14947553 on ECA30; at position 28361747 on ECA17, at position 14648590 on ECA23, at position 14649864 on ECA23, at position 15068782 on ECA30, at position 35731283 on ECA1, at position 20652865 on ECA23, at position 28540291 on ECA17, at position 15055793 on ECA30, at position 35731849 on ECA1, at position 35729338 on ECA1, at position 14936139 on ECA30, at position 20662320 on ECA23, at position 35720250 on ECA1, at position 35721326 on ECA1 and at position 35726345 on ECA1.

25. The method of claim 22, wherein the panel comprises two or more SNPs selected from the group consisting of a SNP located at position 14640812 on equine (ECA) chromosome 23, at position 28347510 on ECA17, at position 14107178 on ECA30, at position 14067984 on ECA30, at position 17945265 on ECA1, at position 81651604 on ECA6 and at position 15044553 on ECA25.

26. The method of any one of claims 22-25, wherein the panel comprises 3 or more SNPs.

27. The method of claim 26, wherein the panel comprises 5 or more SNPs.

28. The method of claim 26, wherein the panel comprises 7 SNPs.

29. The method of any one of claims 20-28, further comprising identifying the horse as having the ability to pace when the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected, the presence of an adenine (A) nucleotide at 15044553 on ECA25 in one or both alleles is detected, the absence of a guanine (G) nucleotide at 14947553 on ECA30 in one or both alleles is detected, the presence of a guanine (G) nucleotide at 28361747 on ECA17 in one or both alleles is detected, the presence of an adenine (A) nucleotide at 14648590 on ECA23 in one or both alleles is detected, the absence of a cytosine (C) nucleotide at 14649864 on ECA23 in one or both alleles is detected, the presence of a guanine (G) nucleotide at 15068782 on ECA30 in one or both alleles is detected, the presence of a thymine (T) nucleotide at 35731283 on ECA1 in one or both alleles is detected, the presence of an adenine (A) nucleotide at 20652865 on ECA23 in one or both alleles is detected, the presence of an adenine (A) nucleotide at 28540291 on ECA17 in one or both alleles is detected, the presence of a cytosine (C) nucleotide at 15055793 on ECA30 in one or both alleles is detected, the presence of a guanine (G) nucleotide at 35731849 on ECA1 in one or both alleles is detected, the presence of a thymine (T) nucleotide at 35729338 on ECA1 in one or both alleles is detected, the presence of an adenine (A) nucleotide at 14936139 on ECA30 in one or both alleles is detected, the presence of an adenine (A) nucleotide at 20662320 on ECA23 in one or both alleles is detected, the presence of a cytosine (C) nucleotide at 35720250 on ECA1 in one or both alleles is detected, the presence of a cytosine (C) nucleotide at 35721326 on ECA1 in one or both alleles is detected and/or the presence of a thymine (T) nucleotide at 35726345 on ECA1 in one or both alleles is detected.

30. The method of claim 29, wherein the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and/or the presence of an adenine (A) nucleotide at 15044553 on ECA25 in one or both alleles is detected.

31. The method of claim 29 or 30, further comprising selecting the identified horse for training or breeding.

32. The method of any one of claims 20-31, further comprising using a learning statistical classifier system to analyze the SNP genotypes.

33. The method of claim 32, wherein the learning statistical classifier system is a Random Forest system.

34. The method of claim 32 or 33, wherein the panel has a misclassification error of less than 10%.

35. The method of claim 34, wherein the panel has a misclassification error of less than 1%.

36. The method of any one of claims 1-35, wherein the assaying step comprises contacting the sample with at least one oligonucleotide probe to form at least one hybridized nucleic acid product.

37. The method of claim 36, further comprising detecting the at least one hybridized nucleic acid product, wherein the detection is performed by polymerase chain reaction (PCR), allele specific hybridization, the 3′exonuclease assay (Taqman assay), fluorescent dye and quenching agent-based PCR assay, allele-specific restriction enzymes (RFLP-based techniques), direct sequencing, the oligonucleotide ligation assay (OLA), pyrosequencing, the invader assay, minisequencing, DHPLC-based techniques, single strand conformational polymorphism (SSCP), allele-specific PCR, denaturating gradient gel electrophoresis (DGGE), temperature gradient gel electrophoresis (TGGE), chemical mismatch cleavage (CMG), heteroduplex analysis based system, techniques based on mass spectroscopy, invasive cleavage assay, polymorphism ratio sequencing (PRS), microarrays, a rolling circle extension assay, HPLC-based techniques, extension based assays, ARMS (Amplification Refractory Mutation System), ALEX (Amplification Refractory Mutation Linear Extension), SBCE (Single base chain extension), molecular beacon assays, invader (Third wave technologies), ligase chain reaction assays, 5′-nuclease assay-based techniques, hybridization capillary array electrophoresis (GAE), or solid phase hybridization (dot blot, reverse dot blot, chips).

38. The method of claim 37, further comprising amplifying the at least one hybridized nucleic acid product.

39. The method of claim 38, wherein the at least one amplified nucleic acid product comprises a SNP located at nucleotide position 14640812 on ECA23, nucleotide position 28347510 on ECA17, nucleotide position 14107178 on ECA30, nucleotide position 14067984 on ECA30, nucleotide position 17945265 on ECA1, nucleotide position 81651604 on ECA6 or nucleotide position 15044553 on ECA25.

40. The method of any one of claims 1-39, wherein the absence of a guanine (G) nucleotide at 14640812 on ECA23 in one or both alleles is detected.

41. The method of claim 40, wherein the absence of a guanine (G) nucleotide at 14640812 on ECA23 in both alleles is detected.

42. The method of any one of claims 1-41, wherein the presence of a guanine (G) nucleotide at 28347510 on ECA17 in one or both alleles is detected.

43. The method of claim 42, wherein the presence of a guanine (G) nucleotide at 28347510 on ECA17 in both alleles is detected.

44. The method of any one of claims 1-43, wherein the absence of an adenine (A) nucleotide at 14107178 on ECA30 in one or both alleles is detected.

45. The method of claim 44, wherein the absence of an adenine (A) nucleotide at 14107178 on ECA30 in both alleles is detected.

46. The method of any one of claims 1-45, wherein the presence of a guanine (G) nucleotide at 14067984 on ECA30 in one or both alleles is detected.

47. The method of claim 46, wherein the presence of a guanine (G) nucleotide at 14067984 on ECA30 in both alleles is detected.

48. The method of any one of claims 1-47, wherein the presence of a cytosine (C) nucleotide at 17945265 on ECA1 in one or both alleles is detected.

49. The method of claim 48, wherein the presence of a cytosine (C) nucleotide at 17945265 on ECA1 in both alleles is detected.

50. The method of any one of claims 1-49, wherein the absence of a thymine (T) nucleotide at 81651604 on ECA6 in one or both alleles is detected.

51. The method of claim 50, wherein the absence of a thymine (T) nucleotide at 81651604 on ECA6 in both alleles is detected.

52. The method of any one of claims 1-51, wherein the presence of an adenine (A) nucleotide at 15044553 on ECA25 in one or both alleles is detected.

53. The method of claim 52, wherein the presence of an adenine (A) nucleotide at 15044553 on ECA25 in both alleles is detected.

54. The method of any one of claims 1-39, wherein the absence of a guanine (G) nucleotide at 14640812 on ECA23 in one or both alleles is detected, the presence of a guanine (G) nucleotide at 28347510 on ECA17 in one or both alleles is detected, the absence of an adenine (A) nucleotide at 14107178 on ECA30 in one or both alleles is detected, the presence of a guanine (G) nucleotide at 14067984 on ECA30 in one or both alleles is detected, the presence of a cytosine (C) nucleotide at 17945265 on ECA1 in one or both alleles is detected, the absence of a thymine (T) nucleotide at 81651604 on ECA6 in one or both alleles is detected and the presence of an adenine (A) nucleotide at 15044553 on ECA25 in one or both alleles is detected.

55. The method of any one of claims 1-39, wherein the absence of a guanine (G) nucleotide at 14640812 on ECA23 in both alleles is detected, the presence of a guanine (G) nucleotide at 28347510 on ECA17 in both alleles is detected, the absence of an adenine (A) nucleotide at 14107178 on ECA30 in both alleles is detected, the presence of a guanine (G) nucleotide at 14067984 on ECA30 in both alleles is detected, the presence of a cytosine (C) nucleotide at 17945265 on ECA1 in both alleles is detected, the absence of a thymine (T) nucleotide at 81651604 on ECA6 in both alleles is detected and the presence of an adenine (A) nucleotide at 15044553 on ECA25 in both alleles is detected.

56. The method of any one of claims 1-55, wherein the horse is a gaited breed.

57. The method of claim 56, wherein the horse is selected from the group consisting of American Saddlebred, Campolina, Icelandic horse, Kentucky Mountain Saddle Horse, Mangalarga Marchador, Marwari horse, Missouri Foxtrotter, Paso Fino, Racking horse, Rocky Mountain horse, Spotted Saddle horse, Standardbred, Tennessee Walker and Walkaloosa.

58. The method of claim 57, wherein the horse is a Standardbred.

59. A nucleic acid comprising a single nucleotide polymorphism (SNP) described herein.

60. A nucleic acid comprising a single nucleotide polymorphism (SNP), wherein the SNP is selected from the group consisting of a SNP at 14640812 on ECA23, a SNP at 28347510 on ECA17, a SNP at 14107178 on ECA30, a SNP at 14067984 on ECA30, a SNP at 17945265 on ECA1, a SNP at 15044553 on ECA25, a SNP at 14947553 on ECA30, a SNP at 28361747 on ECA17, a SNP at 14648590 on ECA23, a SNP at 14649864 on ECA23, a SNP at 15068782 on ECA30, a SNP at 35731283 on ECA1, a SNP at 20652865 on ECA23, a SNP at 28540291 on ECA17, a SNP at 15055793 on ECA30, a SNP at 35731849 on ECA1, a SNP at 35729338 on ECA1, a SNP at 14936139 on ECA30, a SNP at 20662320 on ECA23, a SNP at 35720250 on ECA1, a SNP at 35721326 on ECA1 and a SNP at 35726345 on ECA1.

61. The nucleic acid of claim 60, wherein the SNP is selected from the group consisting of a SNP at 14640812 on ECA23, a SNP at 28347510 on ECA17, a SNP at 14107178 on ECA30, a SNP at 14067984 on ECA30, a SNP at 17945265 on ECA1 and a SNP at 15044553 on ECA25.

62. A kit for identifying a horse that has the ability to pace comprising:
1) at least one oligonucleotide probe capable of forming a hybridized nucleic acid with a SNP or a nucleic acid region flanking a SNP, wherein the SNP is selected from the group consisting of a SNP at 14640812 on ECA23, a SNP at 28347510 on ECA17, a SNP at 14107178 on ECA30, a SNP at 14067984 on ECA30, a SNP at 17945265 on ECA1, a SNP at 81651604 at ECA6, a SNP at 15044553 on ECA25, a SNP at 14947553 on ECA30, a SNP at 28361747 on ECA17, a SNP at 14648590 on ECA23, a SNP at 14649864 on ECA23, a SNP at 15068782 on ECA30, a SNP at 35731283 on ECA1, a SNP at 20652865 on ECA23, a SNP at 28540291 on ECA17, a SNP at 15055793 on ECA30, a SNP at 35731849 on ECA1, a SNP at 35729338 on ECA1, a SNP at 14936139 on ECA30, a SNP at 20662320 on ECA23, a SNP at 35720250 on ECA1, a SNP at 35721326 on ECA1 and a SNP at 35726345 on ECA1; and
2) instructions for identifying the horse as having the ability to pace based on the horse's genotype at the SNP.

63. The kit of claim 62, wherein the SNP is selected from the group consisting of a SNP at 14640812 on ECA23, a SNP at 28347510 on ECA17, a SNP at 14107178 on ECA30, a SNP at 14067984 on ECA30, a SNP at 17945265 on ECA1, a SNP at 81651604 at ECA6 and a SNP at 15044553 on ECA25.


SEQUENCE LISTING (7 sequences)

SEQ ID NO: 1 - DNA, 301 bp, Equus sp.
ctcacatccaaattttgtgatattgtgctggcttgagctccaggggagctggctctggtaaggaaggcttcagatgccctcattgtggtgctaactgcaagaagagccccttgagccccttggatgcagagctgcagtgttcctgtgagarcctgtaagggacagacaagtgctctgtgggggcggggagtcgtaatggggaatataaaacacacacaccttgcataaggccttacatttcatcagtgagtactgtgggcacatgtgttattgcagagaaacatcactagctggagcacct

SEQ ID NO: 2 - DNA, 301 bp, Equus sp.
ctgctatcatacccatctaaagggactttcagcaagttgaaacaagacttttaacagactgtctttgccttttggtaggcacaatcattggcagcatcactttcaaccagacagctactgcggatttctcgtcggctctcacagtaccctratgaaaatcttcacagtgctgttactaaagcctgcctttccaggtaaccatattctcctttctcactgaattcaagtttctgggtctttgctgaaacagtaacataagtttaattgatagtctgtactcacaggtctgcaaaaagtattg

SEQ ID NO: 3 - DNA, 301 bp, Equus sp.
aggtttgctactaacactgtcacatttccagttgatgagaaaggaaacaagcaaaaaatatgcagggtcccctctgtccccgcctgcccctcctcgatatggtgatatgtatgtcaactcccgaacagaatattatgtttcaccgtagatmaaaaacatccccatggatcattttcttttcaaatctaaaatgtaaagagtgaatgctgacggctctctcctttattttcctgtccaccaacaaaaggaatcattgatacaaaggccgctctgactgaggagtttagttttcagaatctac

SEQ ID NO: 4 - DNA, 301 bp, Equus sp.
ttcatcttggtcccaggatccagccccaccttctccctcccacccgcgcggtgcagagcgagggtgcaaaacctttgctctcggcgacagcctcctgcctccctctcggggccagcttctccccgaggtcagcctctgccacctgctttckcagcactgcgttcccttgaagcagctacctcaatctgcttctgtttccacccccaccgcgaacctccccaccaaccccacccgccaacttgggagcaagcttcgttcaccattctacctccaaggctagcaacagggctggcctgtagtg

SEQ ID NO: 5 - DNA, 301 bp, Equus sp.
atcttttaaaaaacacaaatctgatcatgtcaccctcttgcagaaatcccctgttccttgtataaaagccaaatctcacctcacagcacgttctccacatccttcttcccctcgctcagggtgttgcagcagcctggcgttccttcagctsctcaagagtatcattattcctcttgctttaaggccttgacagttcccttgacctggaatgttctcatcctttctcacctagtaaagccatacttgtctttcagatctcaagctcaattttcactcttaagaaagctttctttaattcctc

SEQ ID NO: 6 - DNA, 301 bp, Equus sp.
cacatgactgccagatgagggcaagagaacccttaatgggtatggggtttcctcttagggtgatgaaaatgttttggaaatagaggtgatggtcgggcaacatcatgaatgtactaaatgccactgaactgtacactttacagtggttaaktttattttatgtgagttttagctcgatggcaagaagagcgaacatgcagggctggtgtgcctgtgtggaaacagagacagcaggggtacaaagtttcaaggaggccagctaaaacacagcagcaggtcgagagacatgtgagatggaggg

SEQ ID NO: 7 - DNA, 301 bp, Equus sp.
cattccttggcttctggctgcgtgactccaatttctgcctctgtcttcccatggccttcccctctgggtctgtgtgtcaccaatcttcctctcttttctctgataaagacaccagtcgttaggtttagagcccaccctaaatctagaatgwtctcactgcaagacccttaactttattacatctgtaaagaccctatttccaaataaggtcatattagcaggtactgaggattgggatttgcacctatcttttggggactattaaagccactattgttgtcattattattattttcagttc